(19) United States
(12) Patent Application Publication
(10) Pub. No.: US 2013/0302442 A1
(43) Pub. Date: Nov. 14, 2013

(54) METHODS OF USING (4S,4AS,5AR,12AS)-4-DIMETHYLAMINO-3,10,12,12A-TETRAHYDROXY-7-[(METHOXY(METHYL)AMINO)-METHYL]-1,11-DIOXO-1,4,4A,5,5A,6,11,12A-OCTAHYDRO-NAPHTHACENE-2-CARBOXYLIC ACID AMIDE

(71) Applicants: Paratek Pharmaceuticals, Inc., Boston, MA (US); Warner Chilcott Company, LLC, Fajardo, PR (US)

(72) Inventors: Catherine Coulter, Ballymena (GB); Sean M. Johnston, Doylestown, PA (US); Farzaneh Seyedi, Mansfield, MA (US); Tina M. deVries, Long Valley, NJ (US)

(73) Assignees: Paratek Pharmaceuticals, Inc., Boston, MA (US); Warmer Chilcott Company, LLC, Fajardo, PR (US)

(21) Appl. No.: 13/893,717

(22) Filed: May 14, 2013

Related U.S. Application Data
(60) Provisional application No. 61/646,754, filed on May 14, 2012.

(51) Int. Cl. A61K 31/65; A61K 45/06

(52) U.S. Cl. CPC A61K 31/65; A61K 45/06
USPC 424/653; 514/152; 514/154

(57) ABSTRACT

A method of treating a bacterial infection, e.g., a bacterial infection with methicillin resistant Staphylococcus aureus, Helicobacter pylori, Chlamydia trachomatis, or Chlamydia pneumonia, comprising administering to a subject (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt thereof is disclosed. More specifically, a method of treating a bacterial infection comprising administering to a subject crystalline mono hydrochloride salt, crystalline mono mesylate salt or crystalline mono sulfate salt of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide is disclosed.

### CROSS-REFERENCE TO RELATED APPLICATIONS

This application claims the benefit of priority from U.S. Provisional Application No. 61/646,754, filed May 14, 2012, the content of which is incorporated by reference herein in its entirety.

### FIELD OF THE INVENTION

The instant disclosure relates to methods of treating a bacterial infection, or conditions associated with such infections (e.g., peptic ulcers and Chlamydia), comprising administering to a subject (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt thereof, wherein the bacterial infection is caused by a bacteria selected from the group consisting of methicillin-resistant Staphylococcus aureus, Helicobacter pylori, Chlamydia trachomatis and Chlamydia pneumoniae. In certain embodiments of the invention, a crystalline mono hydrochloride, mono mesylate, or mono sulfate salt of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide is administered.

### BACKGROUND OF THE INVENTION

Tetracyclines are known “broad spectrum” antibiotics and have become widely used for therapeutic purposes. Tetracyclines have been found to be highly effective pharmacologically against rickettsiae; a number of gram-positive and gram-negative bacteria; and the agents responsible for lymphogranuloma venereum, inclusion conjunctivitis, and psittacosis. The first use of tetracycline antibiotics dates as far back as 1948. Examples of pharmaceutically active tetracycline and tetracycline analogue compositions may be found in U.S. Pat. Nos. 2,980,584; 2,990,331; 3,062,717; 3,165,531; 3,454,697; 3,557,280; 3,674,859; 3,957,980; 4,018,889; 4,024,272; and 4,126,680. Tetracyclines may also be used to treat inflammatory skin disorders, including dermatitis, psoriasis, pyoderma gangrenosum, acne and rosacea.

Methicillin-resistant Staphylococcus aureus (MRSA) is a strain of S. aureus bacteria that is resistant to some commonly used antibiotics. Commonly, MRSA infections occur in hospital or other health care facility setting (hospital acquired MRSA) to people with compromised immune systems, but sometimes they can occur in otherwise healthy individuals outside the hospital (community associated MRSA). Most MRSA infections involve the skin, but some may be severe and spread to bloodstream, heart or lungs, urine, or at the site of a recent surgery. MRSA is commonly treated with antibiotics such as doxycycline, clindamycin, linezolid, or a combination of trimethoprim/sulfamethoxazole (TMP/SXT).

Chlamydia is a sexually transmitted disease caused by bacteria Chlamydia trachomatis. It is the most common sexually transmitted disease in the United States and is the cause of more than 3 million cases of cervicitis and urethritis every year. Chlamydia infections in women often lead to inflammation of the cervix, and if left untreated, may lead to infertility. Infections in men often lead to inflammation of the urethra. Early antibiotic treatment of Chlamydia may prevent the development of these long-term complications.

Chlamydia pneumoniae is a small gram negative bacterium and a frequent cause of community-acquired respiratory infections, including pneumonia and bronchitis, in adults and children.

Peptic ulcer is a disease characterized by inflammation of the lining of the stomach and duodenum, where the protective lining of these organs breaks down and allows for irritation caused by acids produced in the stomach. A common cause of protective lining breakdown is an infection by Helicobacter pylori bacteria. Typical treatment includes a combination of antibiotics and other agents, including proton pump inhibitors (e.g., omeprazole), and/or bismuth.

After the widespread use of tetracyclines for both major and minor illnesses and diseases led to resistance to these antibiotics, substituted tetracycline compounds were developed to treat bacterial infections, inflammation, neoplasms, and other conditions. The term “tetracycline compound” includes many compounds with a similar ring structure to tetracycline. Examples of these tetracycline compounds include: chlortetracycline, doxycycline, minocycline, oxytetracycline, demeclocycline, methacycline, sancycline, chelocardin, rolitetracycline, lymecycline, apicycline; clomocycline, guamecycline, meglucycline, mepylcycline, penimepicycline, pipacycline, etamocycline, penimocycline. For example, substituted tetracycline compounds have been disclosed in WO 2008/079339 and WO 2008/079363.

One substituted tetracycline compound is (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide, described in U.S. Patent Application Publication Nos. 2008/0312193 and 2010/0305072.

In part, because of the developed resistance to certain antibiotics, there exists a need in the art for new tetracycline antibiotics for the treatment of illnesses and diseases.

The present invention is directed to a novel method for treating a bacterial infection and conditions associated with such infections (e.g., peptic ulcer) comprising administering to a subject (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt thereof.

### SUMMARY OF THE INVENTION

The present invention is directed to methods of treating a bacterial infection, wherein the bacteria is selected from the group consisting of methicillin resistant Staphylococcus aureus (MRSA), Helicobacter pylori, Chlamydia trachomatis, and Chlamydia pneumoniae, comprising administering to a subject a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt thereof. In certain embodiments, a crystalline salt of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide is administered, and preferably, the crystalline salt may be selected from the group consisting of mono hydrochloride, mono mesylate and mono sulfate.

In a certain embodiment, the MRSA is a community associated MRSA (MRSA-CA) or hospital acquired MRSA (MRSA-HA).

In another aspect, the invention is directed to a method of treating peptic ulcer comprising administering to a subject a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt thereof. In one embodiment, the salt is a crystalline salt, which may be preferably selected from the group consisting of mono hydrochloride salt, mono mesylate salt and mono sulfate salt. In one embodiment, the method of treating peptic ulcer further comprises administering at least one additional active ingredient, e.g., a proton pump inhibitor and/or bismuth.

### BRIEF DESCRIPTION OF THE FIGURES

FIG. 1 shows X-ray powder diffraction (XRPD) analysis of crystalline mono hydrochloride salt of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide after synthesis and after storage for 7 days at 40° C. and 75% relative humidity (RH).

FIG. 2 is a differential scanning calorimetry (DSC) curve of crystalline mono hydrochloride salt of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide after synthesis.

FIG. 3 is a thermo-gravimetric analysis (TGA) curve of crystalline mono hydrochloride salt of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide after synthesis.

FIG. 4 shows XRPD analysis of crystalline mono mesylate salt of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide after synthesis and after storage for 7 days at 40° C. and 75% RH.

FIG. 5 is a DSC curve of crystalline mono mesylate salt of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide after synthesis.

FIG. 6 is a TGA of crystalline mono mesylate salt of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide after synthesis.

FIG. 7 shows XRPD analysis of crystalline mono sulfate salt of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide after synthesis and after storage for 7 days at 40° C. and 75% RH.

FIG. 8 is a DSC curve of crystalline mono sulfate salt of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide after synthesis.

FIG. 9 is a TGA of crystalline mono sulfate salt of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide after synthesis.

FIG. 10 shows XRPD analysis of amorphous bis hydrochloride salt of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide.

FIG. 11 is a TGA curve and DSC curve overlaid of amorphous bis hydrochloride salt of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide.

### DETAILED DESCRIPTION OF THE INVENTION

### Methods of the Invention

The first embodiment of the invention is directed to a method for treating a bacterial infection, wherein the bacteria is methicillin resistant Staphylococcus aureus (MRSA), Helicobacter pylori, or Chlamydia trachomatis, comprising administering to a subject a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt thereof. In a preferred embodiment, the method comprises administering to a subject a therapeutically effective amount of a crystalline salt of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide. Preferably, the crystalline salt may be selected from the group consisting of mono hydrochloride, mono mesylate and mono sulfate. These crystalline salts are fully described in co-pending U.S. application Ser. No. 13/471,275, the contents of which are incorporated herein by reference in its entirety.

In certain embodiments, the amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt thereof employed is between about 10 mg and about 2000 mg, and preferably between about 25 mg and about 500 mg. In a certain embodiment, (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt thereof is administered at least once monthly, preferably, weekly, more preferably, bi-weekly, and most preferably, daily.

In one embodiment, the invention is directed to a method of treating methicillin resistant Staphylococcus aureus (MRSA) comprising administering to a subject a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt thereof. In an embodiment thereof, wherein the invention is directed to a method of treating MRSA, the MRSA is hospital acquired (MRSA-HA) or community associated (MRSA-CA).

In another embodiment, the invention is directed to a method of treating Helicobacter pylori comprising administering to a subject a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt thereof. In a further embodiment, the invention is directed to a method of treating Chlamydia trachomatis comprising administering to a subject a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt thereof.

The term “treating” as used herein includes therapeutic and/or prophylactic treatment of the bacterial infection or other conditions described herein. The treatment includes the diminishment or alleviation of at least one symptom associated with the bacterial infection or at least one symptom associated with another condition described herein.

The term “therapeutically effective amount” as used herein means an amount of a compound or composition high enough to significantly positively modify the symptoms and/or condition to be treated, but low enough to avoid serious side effects (at a reasonable risk/benefit ratio), within the scope of sound medical judgment. The therapeutically effective amount of active ingredient for use in the method of the invention herein will vary with the particular condition being treated, the age and physical condition of the patient to be treated, the severity of the condition, the duration of the treatment, the nature of concurrent therapy, the particular active ingredient being employed, the particular pharmaceutically-acceptable excipients utilized, and like factors within the knowledge and expertise of a skilled physician or veterinarian. Various suitable therapeutically effective amounts are described above.

The term “subject” as used herein is an animal. “Subject” includes, without limitation, a human, mouse, rat, guinea pig, dog, cat, horse, cow, pig, monkey, chimpanzee, baboon, or rhesus monkey. In one embodiment, “subject” is a mammal. In another embodiment, “subject” is a human.

The phrase “pharmaceutically acceptable salt” of a compound as used herein means a salt that is pharmaceutically acceptable and that possesses the desired pharmacological activity of the parent compound. Pharmaceutically acceptable salts include salts of acidic or basic groups present in a compound of the invention. Pharmaceutically acceptable acid addition salts include, but are not limited to, hydrochloride, hydrobromide, hydroiodide, nitrate, mesylate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Suitable base salts include, but are not limited to, aluminum, calcium, lithium, magnesium, potassium, sodium, zinc, and diethanolamine salts. Preferably, the pharmaceutically acceptable salt is a crystalline salt. Even more preferably, the pharmaceutically acceptable salt is a crystalline salt selected from mono hydrochloride, mono mesylate, and mono sulfate.

A certain embodiment is directed to the method for treating methicillin resistant Staphylococcus aureus (MRSA) comprising administering to a subject a therapeutically effective amount of the crystalline mono hydrochloride salt of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide. In an embodiment thereof, the amount of the mono hydrochloride salt employed is between about 10 mg and about 2000 mg, and preferably between about 25 mg and about 500 mg. In a certain embodiment, the mono hydrochloride salt is administered at least once monthly, preferably, weekly, more preferably, bi-weekly, and most preferably, the mono hydrochloride salt is administered daily.

Another embodiment is directed to the method for treating methicillin resistant Staphylococcus aureus (MRSA) comprising administering to a subject a therapeutically effective amount of the crystalline mono mesylate salt of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide. In certain embodiments thereof, the amount of crystalline mono mesylate salt employed is between about 10 mg and about 2000 mg, and preferably between about 25 mg and about 500 mg. In a certain embodiment, the mono mesylate salt is administered at least once monthly, preferably, weekly, more preferably, bi-weekly, and most preferably, the mono mesylate salt is administered daily.

A further embodiment is directed to the method for treating methicillin resistant Staphylococcus aureus (MRSA) comprising administering to a subject a therapeutically effective amount of the crystalline mono sulfate salt of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide. In certain embodiments, the amount of crystalline mono sulfate salt employed is between about 10 mg and about 2000 mg, and preferably between about 25 mg and about 500 mg. In a certain embodiment, the mono sulfate salt is administered at least once monthly, preferably, weekly, more preferably, bi-weekly, and most preferably, the mono sulfate salt is administered daily.

A certain embodiment is directed to the method for treating Chlamydia trachomatis comprising administering to a subject a therapeutically effective amount of the crystalline mono hydrochloride salt of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide. In an embodiment thereof, the amount of the mono hydrochloride salt employed is between about 10 mg and about 2000 mg, and preferably between about 25 mg and about 500 mg. In a certain embodiment, the mono hydrochloride salt is administered at least once monthly, preferably, weekly, more preferably, bi-weekly, and most preferably, the mono hydrochloride salt is administered daily.

Another embodiment is directed to the method for treating Chlamydia trachomatis comprising administering to a subject a therapeutically effective amount of the crystalline mono mesylate salt of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide. In certain embodiments thereof, the amount of crystalline mono mesylate salt employed is between about 10 mg and about 2000 mg, and preferably between about 25 mg and about 500 mg. In a certain embodiment, the mono mesylate salt is administered at least once monthly, preferably, weekly, more preferably, bi-weekly, and most preferably, the mono mesylate salt is administered daily.

A further embodiment is directed to the method for treating Chlamydia trachomatis comprising administering to a subject a therapeutically effective amount of the crystalline mono sulfate salt of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide. In certain embodiments, the amount of crystalline mono sulfate salt employed is between about 10 mg and about 2000 mg, and preferably between about 25 mg and about 500 mg. In a certain embodiment, the mono sulfate salt is administered at least once monthly, preferably, weekly, more preferably, bi-weekly, and most preferably, the mono sulfate salt is administered daily.

A certain embodiment is directed to the method for treating Helicobacter pylori comprising administering to a subject a therapeutically effective amount of the crystalline mono hydrochloride salt of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide. In an embodiment thereof, the amount of the mono hydrochloride salt employed is between about 10 mg and about 2000 mg, and preferably between about 25 mg and about 500 mg. In a certain embodiment, the mono hydrochloride salt is administered at least once monthly, preferably, weekly, more preferably, bi-weekly, and most preferably, the mono hydrochloride salt is administered daily.

Another embodiment is directed to the method for treating Helicobacter pylori comprising administering to a subject a therapeutically effective amount of the crystalline mono mesylate salt of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide. In certain embodiments thereof, the amount of crystalline mono mesylate salt employed is between about 10 mg and about 2000 mg, and preferably between about 25 mg and about 500 mg. In a certain embodiment, the mono mesylate salt is administered at least once monthly, preferably, weekly, more preferably, bi-weekly, and most preferably, the mono mesylate salt is administered daily.

A further embodiment is directed to the method for treating Helicobacter pylori comprising administering to a subject a therapeutically effective amount of the crystalline mono sulfate salt of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide. In certain embodiments, the amount of crystalline mono sulfate salt employed is between about 10 mg and about 2000 mg, and preferably between about 25 mg and about 500 mg. In a certain embodiment, the mono sulfate salt is administered at least once monthly, preferably, weekly, more preferably, bi-weekly, and most preferably, the mono sulfate salt is administered daily.

A further embodiment of the invention is directed to a method of treating a peptic ulcer comprising administering to a subject a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt thereof. In a preferred embodiment, the method comprises administering to a subject a therapeutically effective amount of a crystalline salt of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide. In an embodiment thereof, the crystalline salt may be selected from the group consisting of mono hydrochloride, mono mesylate and mono sulfate.

A certain embodiment is directed to the method for treating a peptic ulcer comprising administering to a subject a therapeutically effective amount of the crystalline mono hydrochloride salt of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide. In an embodiment thereof, the amount of the mono hydrochloride salt employed is between about 10 mg and about 2000 mg, and preferably between about 25 mg and about 500 mg. In a certain embodiment, the mono hydrochloride salt is administered at least once monthly, preferably, weekly, more preferably, bi-weekly, and most preferably, the mono hydrochloride salt is administered daily.

Another embodiment is directed to the method for treating a peptic ulcer comprising administering to a subject a therapeutically effective amount of the crystalline mono mesylate salt of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide. In certain embodiments thereof, the amount of crystalline mono mesylate salt employed is between about 10 mg and about 2000 mg, and preferably between about 25 mg and about 500 mg. In a certain embodiment, the mono mesylate salt is administered at least once monthly, preferably, weekly, more preferably, bi-weekly, and most preferably, the mono mesylate salt is administered daily.

A further embodiment is directed to the method for treating a peptic ulcer comprising administering to a subject a therapeutically effective amount of the crystalline mono sulfate salt of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide. In certain embodiments, the amount of crystalline mono sulfate salt employed is between about 10 mg and about 2000 mg, and preferably between about 25 mg and about 500 mg. In a certain embodiment, the mono sulfate salt is administered at least once monthly, preferably, weekly, more preferably, bi-weekly, and most preferably, the mono sulfate salt is administered daily.

In a certain embodiment, the method of treating a peptic ulcer further comprises administering to the subject at least one additional active ingredient, including but not limited to a proton pump inhibitor and/or bismuth. Various suitable additional active ingredients to treat peptic ulcers are described in Bertram G. Katzung, “Basic and Clinical Pharmacology,” 1064-68 (8^{th} ed., 2001), incorporated by reference herein in its entirety.

An additional embodiment of the invention is directed to a method of treating Chlamydia pneumoniae comprising administering to a subject a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt thereof. In a preferred embodiment, the method comprises administering to a subject a therapeutically effective amount of a crystalline salt of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide. Preferably, the crystalline salt is selected from the group consisting of mono hydrochloride, mono mesylate and mono sulfate.

A certain embodiment is directed to the method for treating Chlamydia pneumoniae comprising administering to a subject a therapeutically effective amount of the crystalline mono hydrochloride salt of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide. In an embodiment thereof, the amount of the mono hydrochloride salt employed is between about 10 mg and about 2000 mg, and preferably between about 25 mg and about 500 mg. In a certain embodiment, the mono hydrochloride salt is administered at least once monthly, preferably, weekly, more preferably, bi-weekly, and most preferably, the mono hydrochloride salt is administered daily.

Another embodiment is directed to the method for treating Chlamydia pneumoniae comprising administering to a subject a therapeutically effective amount of the crystalline mono mesylate salt of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide. In certain embodiments thereof, the amount of crystalline mono mesylate salt employed is between about 10 mg and about 2000 mg, and preferably between about 25 mg and about 500 mg. In a certain embodiment, the mono mesylate salt is administered at least once monthly, preferably, weekly, more preferably, bi-weekly, and most preferably, the mono mesylate salt is administered daily.

A further embodiment is directed to the method for treating Chlamydia pneumoniae comprising administering to a subject a therapeutically effective amount of the crystalline mono sulfate salt of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide. In certain embodiments, the amount of crystalline mono sulfate salt employed is between about 10 mg and about 2000 mg, and preferably between about 25 mg and about 500 mg. In a certain embodiment, the mono sulfate salt is administered at least once monthly, preferably, weekly, more preferably, bi-weekly, and most preferably, the mono sulfate salt is administered daily.

### Crystalline Salts

(4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide and pharmaceutically acceptable salts thereof are used in the methods disclosed herein. In certain embodiments, a crystalline mono hydrochloride, crystalline mono mesylate or crystalline mono sulfate salt of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide is used for treating a bacterial infection selected from the group consisting of methicillin-resistant Staphylococcus aureus, Helicobacter pylori, Chlamydia trachomatis and Chlamydia pneumoniae, or conditions associated with such infections.

The term “crystalline” as used herein refers to compounds in a solid state having a periodic and repeating three-dimensional internal arrangement of atoms, ions or molecules characteristic of crystals. The term crystalline does not necessarily mean that the compound exists as crystals, but that it has this crystal-like internal structural arrangement. The term “amorphous” as used herein refers to compounds lacking a crystalline structure: no repeating pattern, only short range order, extensively disordered.

The crystalline salts of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide may be used to treat, prevent, or otherwise ameliorate bacterial, viral, parasitic, and fungal infections; cancer (e.g., prostate, breast, colon, lung melanoma and lymph cancers) and other disorders characterized by unwanted cellular proliferation; arthritis; osteoporosis; diabetes; stroke; acute myocardial infarction; aortic aneurysm; neurodegenerative diseases and other conditions for which tetracycline compounds have been found to be active (see, for example, U.S. Pat. Nos. 5,789,395; 5,834,450; 6,277,061; and 5,532,227, each of which is expressly incorporated herein by reference). In addition, salts of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide can be used to prevent or control important mammalian and veterinary diseases such as rickettsial infections, sexually transmitted infections, respiratory tract infections, bacterial infections, ophthalmic infections, anthrax; may serve as therapy in acute intestinal amebiasis, acne, lyme disease, and peptic ulcer; and may be used for prophylaxis of malaria and the like.

The term “mono hydrochloride salt” as used herein refers to an ionic compound that results from the neutralization reaction of an acid and a base. The ionic compound (herein, HCl) is composed of a cation and an anion so that the compound is neutral.

General methods for analyzing crystalline salts include crystal analysis by X-ray powder diffraction (XRPD), differential scanning calorimetry (DSC) and thermo-gravimetric analysis (TGA).

XRPD analysis as disclosed herein was collected on a Bruker AXS C2 GADDS diffractometer using Cu Kα radiation (40 kV, 40 mA), automated XYZ stage, laser video microscope for auto-sample positioning and a HiStar 2-dimensional area detector. X-ray optics consisted of a single Gael multilayer mirror coupled with a pinhole collimator of 0.3 mm. The software used for data collection was GADDS for WNT 4.1.16 and the data was analyzed and presented using Diffrac Plus EVA v 9.0.0.2 or v 13.0.0.2. Samples were analyzed under ambient conditions as flat plate specimens using powder as received. Approximately 1-2 mg of the sample was lightly pressed on a glass slide to obtain a flat surface. Samples analyzed under non-ambient conditions were mounted on a silicon wafer with a heat conducting compound. The sample was then heated to the appropriate temperature at approximately 20° C. min⁻¹ and subsequently held isothermally for approximately 1 minute before data collection was initiated.

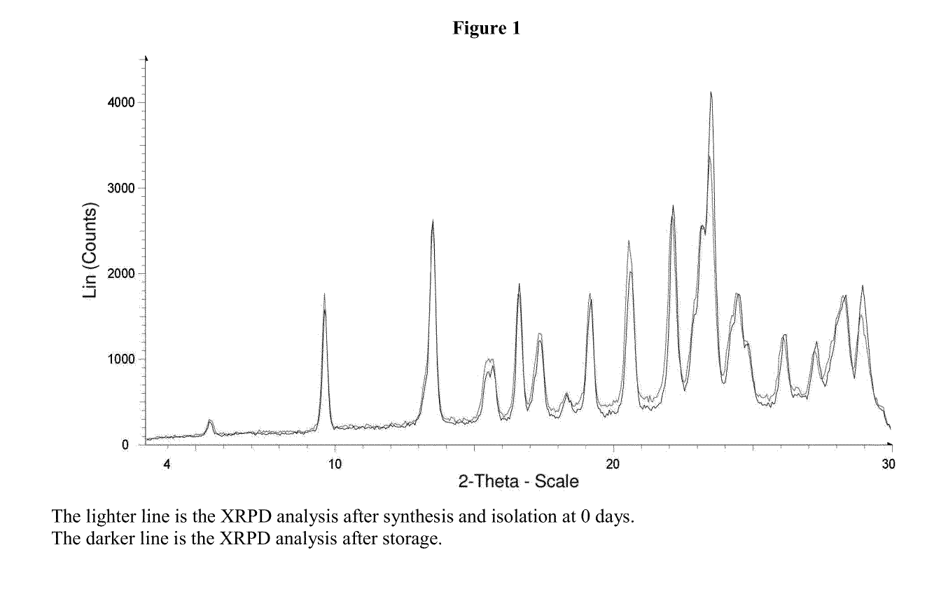

The crystalline mono hydrochloride salt of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide used in the invention has an XRPD pattern substantially as illustrated in FIG. 1 after synthesis of the crystalline salt.

The term “XRPD pattern” as used herein refers to the graphical representation of the data collected by XRPD analysis. XRPD analysis is a technique used to characterize the crystallographic structure, size, and preferred orientation in polycrystalline or powdered solid samples. This diffraction is also used to characterize heterogeneous solid mixtures to determine the percent of crystalline compounds present and can provide structural information on unknown materials.

The terms “substantially” and “about” as used herein in reference to an XPRD pattern refer to the XPRD pattern wherein a listed peak(s) appears within 0.2 degrees 2-theta, including within 0.1 degrees 2-theta of a given 2-theta value.

The crystalline mono hydrochloride salt may have characteristic peaks at diffraction angle 2-theta degrees appearing at least at about 13.4, about 20.5 and about 23.3, as measured by XRPD. More preferably, the crystalline mono hydrochloride salt may have characteristic peaks at diffraction angle 2-theta degrees appearing at least at about 9.5, about 13.4, about 15.5, about 20.5 and about 23.3, as measured by XRPD, and still more preferably, the crystalline mono hydrochloride salt may have characteristic peaks at diffraction angle 2-theta degrees appearing at least at about 9.5, about 13.4, about 15.5, about 16.6, about 19.2, about 20.5, about 22.2, and about 23.3.

The term “characteristic peak” as used herein refers to a peak in the XRPD pattern having an intensity at least 20%, more preferably 40% greater than the baseline noise.

TGA and DSC analysis are used to measure thermal behavior and can be used to distinguish between polymorphs. One polymorphic form may exhibit thermal behavior different from that of the amorphous material or another polymorphic form.

DSC analysis as disclosed herein was collected on a TA Instruments Q2000 equipped with a 50 position auto-sampler. The instrument was calibrated for energy and temperature using certified indium. The calibration for thermal capacity was carried out using sapphire. Typically, 0.5-3.0 mg of each sample, in a pin-holed aluminum pan, was heated at 10° C. min⁻¹ from 25° C. to 250° C. A nitrogen purge at 50 ml·min⁻¹ was maintained over the sample. The instrument control software used was Advantage for Q Series v2.8.0.392 and Thermal Advantage v4.8.3 and the data was analyzed using Universal Analysis v4.4A.

DSC is a thermoanalytical technique in which the difference in the amount of heat required to increase the temperature of a sample and reference is measured as a function of temperature. DSC can be used to measure a number of characteristic properties of a sample, allowing observation of crystallization events. Specifically, with DSC, it is possible to observe small energy changes that occur as matter transitions from a solid to a liquid crystal and from a liquid crystal to an isotropic liquid. The presence of events in the DSC curve can be used to assess the compound's stability, as well as the presence of solvates or hydrates.

TGA is used to determine changes in weight in relation to change in temperature, which may reveal degradation of the compound and the presence of solvates or hydrates. TGA analysis as disclosed herein was collected on a TA Instruments Q500 TGA equipped with a 16 position auto-sampler. The instrument was temperature calibrated using certified Alumel and Nickel. Typically, 5-30 mg of each sample was loaded onto a pre-weighed platinum crucible and aluminum DSC pan and was heated at 10° C. min⁻¹ from ambient temperature to 300° C. A nitrogen purge at 60 ml·min⁻¹ was maintained over the sample. The instrument control and data analysis software used was Advantage for Q Series v2.8.0.392 and Thermal Advantage v4.8.3 and the data was analyzed using Universal Analysis v4.4A.

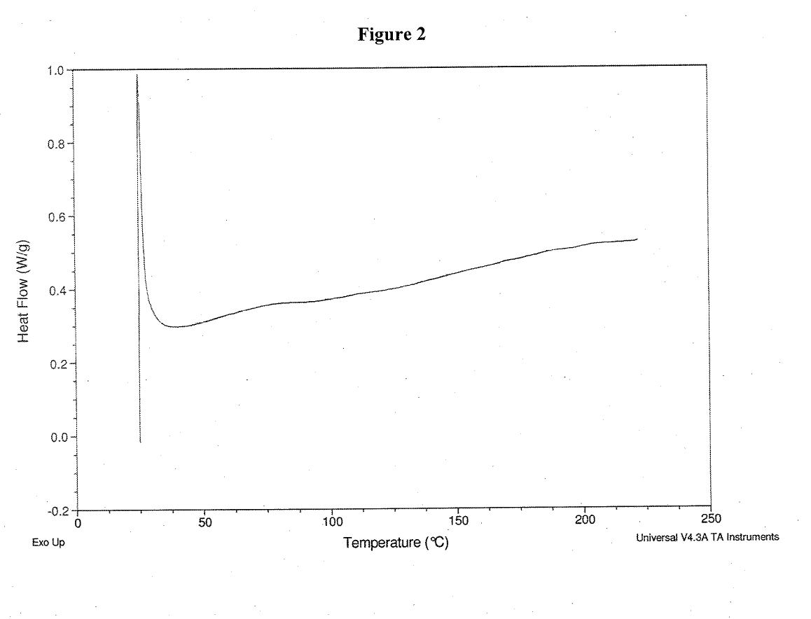

The crystalline mono hydrochloride salt of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide used in the present invention may exhibit a DSC curve substantially as illustrated in FIG. 2, and, preferably, may exhibit no events up to degradation of the crystalline salt.

The term “events” as used herein refers to a change in the sample associated with absorption (endothermic) or evolution (exothermic) of heat causing a change in differential heat flow which is recorded as a peak in the thermogram. Such changes in the sample include decomposition, degradation, and change of form or morphology, solvate or hydrate. The absence of any events indicates that the compound is stable and is in a low energy form.

The term “substantially,” as used herein in reference to DSC curve means the DSC curve demonstrating a peak(s) within 1° C., including within 0.5° C. of a given temperature.

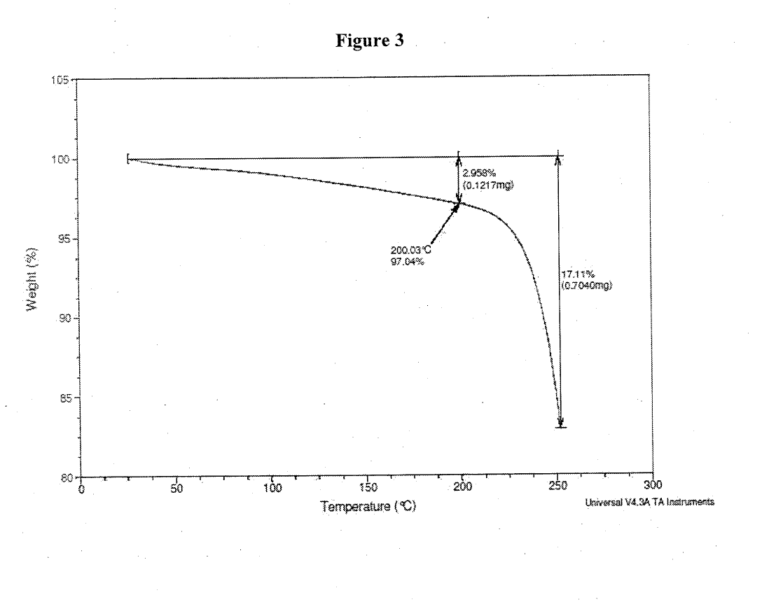

The crystalline mono hydrochloride salt of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide may exhibit a TGA curve substantially as illustrated in FIG. 3, and, preferably, may exhibit a weight loss of about 1% to about 5% from about 30° C. to about 200° C. and a weight loss of about 12% to about 16% from about 200° C. to about 250° C. and, more preferably, a weight loss of about 3% from about 30° C. to about 200° C. and a weight loss of about 14% to about 15% from about 200° C. to about 250° C.

The term “substantially,” as used herein in reference to the TGA curve means the curve demonstrating a percent weight loss within 1%, including within 0.5% of a given value in relation to temperature change.

The term “stable” and “stability” as used herein refers to both the physical form and the chemical purity of the salt. “The salt” as used herein refers to the disclosed crystalline mono hydrochloride, mono mesylate and mono sulfate salts of the present invention.

Ambient conditions, as used herein, means a temperature of about 20° C. to about 25° C. and an RH of about 40%.

In another embodiment, the crystalline mono mesylate salt of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide may be used with the methods of the present invention.

The term “mono mesylate salt” as used herein refers to an ionic compound that results from the neutralization reaction of an acid and a base. The compound is composed of a cation and an anion (herein, CH₃SO₂⁻) so that the compound is neutral.

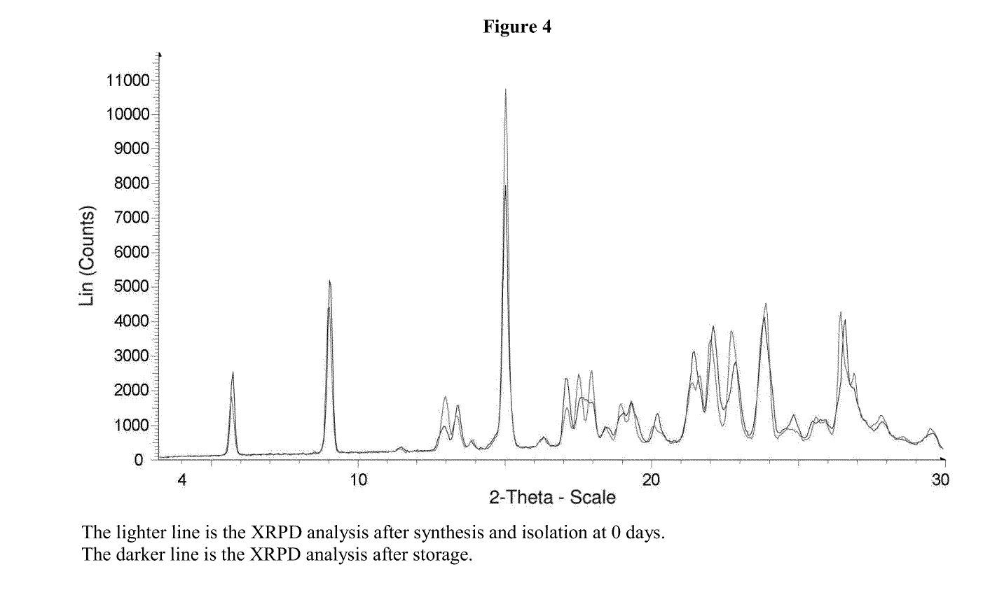

The crystalline mono mesylate salt of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide has an XRPD pattern substantially as illustrated in FIG. 4 after synthesis of the crystalline salt.

The crystalline mono mesylate salt may have characteristic peaks at least appearing at diffraction angle 2-theta degrees appearing at about 9, about 15 and about 23.8, as measured by XRPD. In a preferred embodiment, the crystalline mono mesylate salt may have characteristic peaks at diffraction angle 2-theta degrees appearing at least at about 9, about 15, about 22.7 and about 23.8, as measured by XRPD, and still more preferably, the crystalline mono mesylate salt may have characteristic peaks at diffraction angle 2-theta degrees appearing at least at about 9, about 15, about 22, about 22.7 and about 23.8.

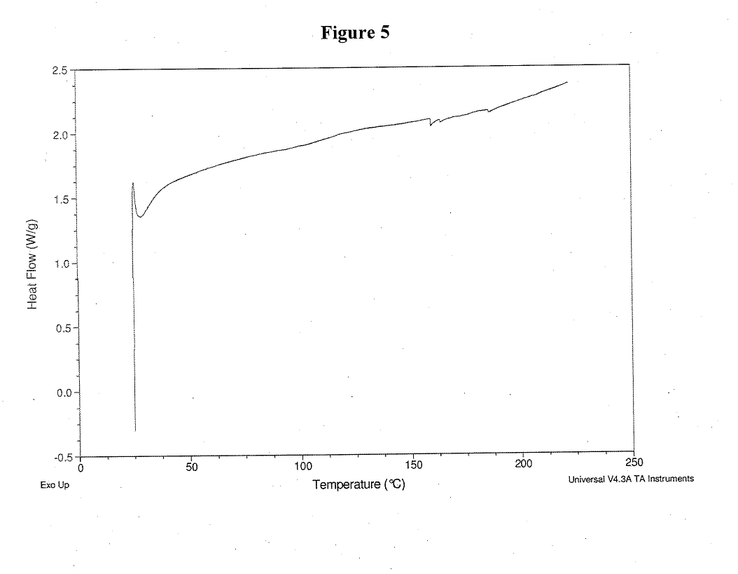

The crystalline mono mesylate salt of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide may exhibit a DSC curve substantially as illustrated in FIG. 5, and, preferably, may exhibit no events up to degradation of the crystalline salt.

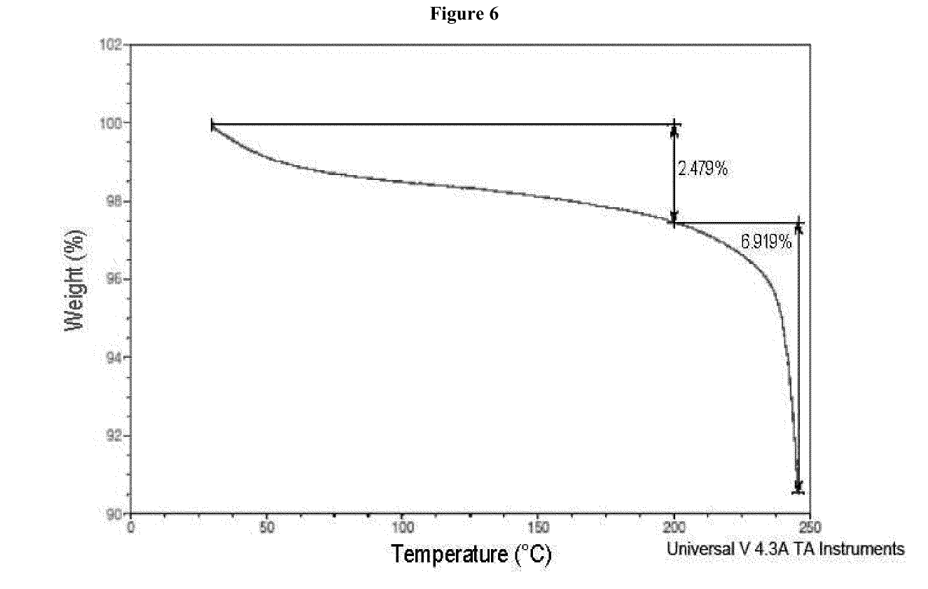

The crystalline mono mesylate salt of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide may exhibit a TGA curve substantially as illustrated in FIG. 6, and, preferably, may exhibit a weight loss of about 1% to about 4% from about 30° C. to about 200° C. and a weight loss of about 3% to about 10% from about 200° C. to about 250° C. and, more preferably, a weight loss of about 2% to about 3% from about 30° C. to about 200° C. and a weight loss of about 6% to about 7% from about 200° C. to about 250° C.

In a further embodiment of the present invention, the crystalline mono sulfate salt of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide may be used with the methods of the present invention.

The term “mono sulfate salt” as used herein refers to an ionic compound that results from the neutralization reaction of an acid and a base. The compound is composed of a cation and an anion (herein, SO₄²⁻) so that the compound is neutral.

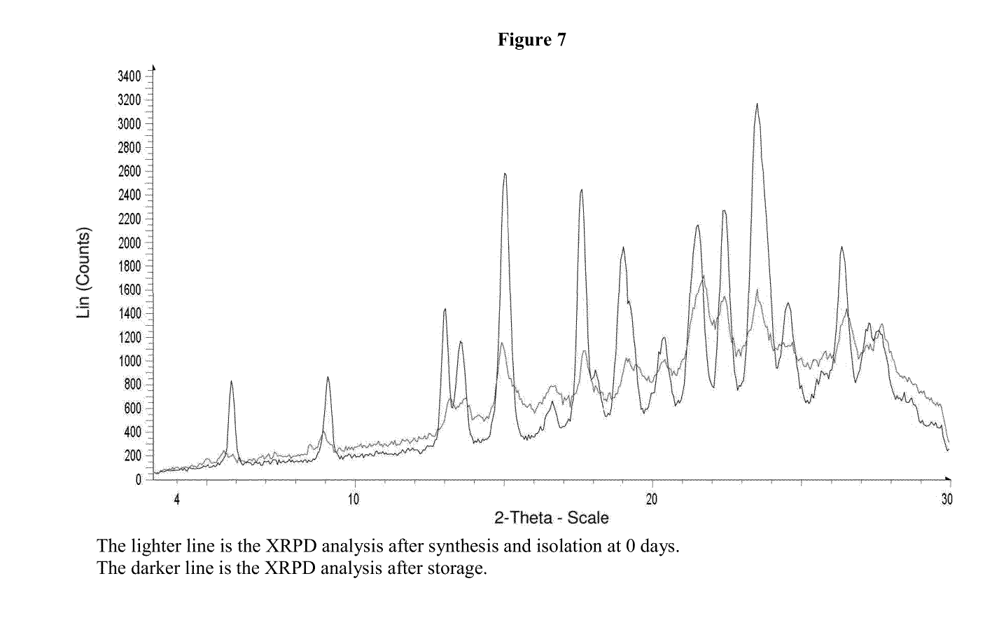

The crystalline mono sulfate salt of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide has an XRPD pattern substantially as illustrated in FIG. 7 after synthesis of the crystalline salt.

The crystalline mono sulfate salt may have characteristic peaks at diffraction angle 2-theta degrees appearing at least at about 15, about 17.8 and about 23.5, as measured by XRPD. In a more preferred embodiment, the crystalline mono sulfate salt may have characteristic peaks at diffraction angle 2-theta degrees appearing at least at about 15, about 17.8, about 22.5 and about 23.5, as measured by XRPD. In a still more preferred embodiment, the crystalline mono sulfate salt may have characteristic peaks at diffraction angle 2-theta degrees appearing at least at about 15, about 17.8, about 19.0, about 22.5 and about 23.5, as measured by XRPD.

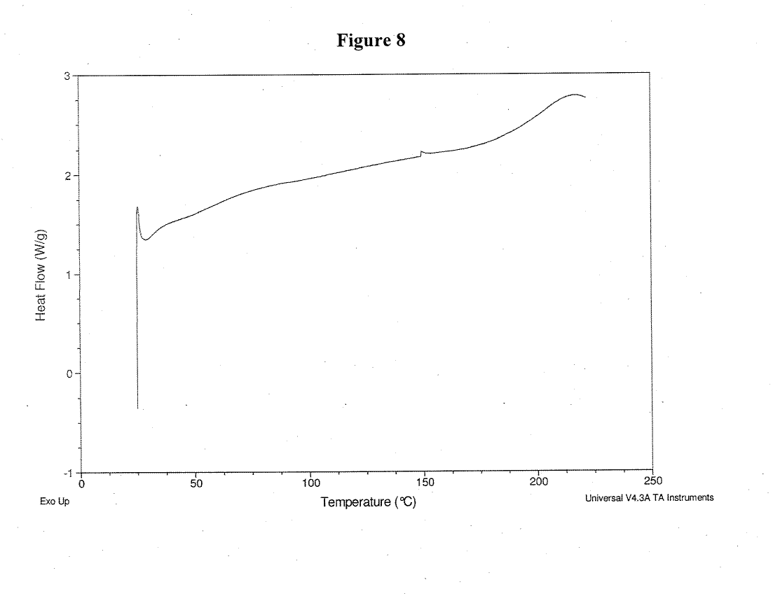

The crystalline mono sulfate salt of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide may exhibit a DSC curve substantially as illustrated in FIG. 8, and, preferably, the crystalline mono sulfate salt analyzed by DSC may exhibit no events up to degradation of the crystalline salt.

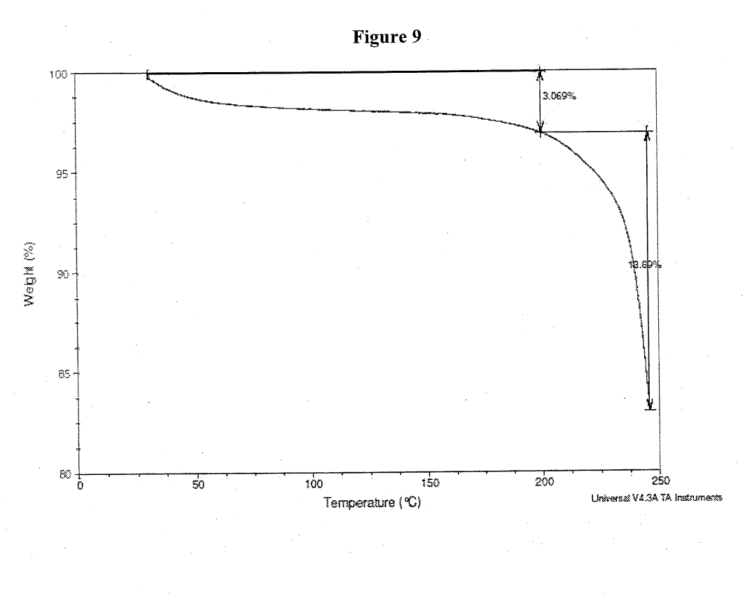

The crystalline mono sulfate salt of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide may exhibit a TGA curve substantially as illustrated in FIG. 9, and, preferably, the crystalline mono sulfate salt analyzed by TGA may exhibit a weight loss of about 1% to about 5% from about 30° C. to about 200° C. and a weight loss of about 12% to about 16% from about 200° C. to about 250° C. and, more preferably, a weight loss of about 3% to about 4% from about 30° C. to about 200° C. and a weight loss of about 13% to about 14% from about 200° C. to about 250° C.

### Pharmaceutical Compositions

One embodiment of the invention is directed to a pharmaceutical composition comprising (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt thereof and a pharmaceutically acceptable excipient for use in treating a bacterial infection selected from the group consisting of methicillin resistant Staphylococcus aureus (MRSA, e.g., MRSA-CA and MRSA-HA), Helicobacter pylori, Chlamydia trachomatis, and Chlamydia pneumoniae, or conditions associated with such infections (e.g., peptic ulcers and Chlamydia). Thus, in one aspect, the pharmaceutical composition of the invention is used for treating peptic ulcers.

In a preferred embodiment, the pharmaceutical composition comprises a crystalline salt of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide, and, preferably, may be selected from the group consisting of mono hydrochloride, mono mesylate and mono sulfate salt.

The pharmaceutical composition of the present invention comprises an effective amount of a crystalline salt, a pharmaceutically acceptable excipient, and, in some embodiments, it may also contain one or more additional active ingredients. The content of crystalline salt in the pharmaceutical composition of the present invention varies depending on the subject of administration, route of administration and target disease, among other variables. The pharmaceutical composition of the present invention may be administered orally, topically (e.g., transdermal, etc.), vaginally, rectally, or parenterally (e.g., intravenous, etc.). Preferably, the pharmaceutical composition of the present invention may be used for treating bacterial infections.

Examples of topical administration of the pharmaceutical composition include transdermal, buccal or sublingual application. For topical applications, the pharmaceutical composition can be suitably admixed in a pharmacologically inert topical carrier, such as a gel, an ointment, a lotion or a cream. Such pharmacologically inert topical carriers include water, glycerol, alcohol, propylene glycol, fatty alcohols, triglycerides, fatty acid esters, or mineral oils. Other possible pharmacologically inert topical carriers are liquid petrolatum, isopropylpalmitate, polyethylene glycol, ethanol 95%, polyoxyethylene monolauriate 5% in water, sodium lauryl sulfate 5% in water, and the like. In addition, materials such as anti-oxidants, humectants, viscosity stabilizers and the like also may be added.

For oral administration, the crystalline salt of the present invention may be administered as a capsule, tablet or granule. Tablets may contain various excipients such as microcrystalline cellulose, sodium citrate, calcium carbonate, dicalcium phosphate and glycine, along with various disintegrants such as starch (and preferably corn, potato or tapioca starch), alginic acid and certain complex silicates, together with granulation binders like polyvinylpyrrolidone, sucrose, gelatin and acacia. In a certain embodiment, the tablet may be film coated. Additionally, lubricating agents such as magnesium stearate, sodium lauryl sulfate and talc are often very useful for tablets. Other solid compositions may also be employed as fillers in gelatin capsules; preferred materials in this connection also include lactose or milk sugar as well as high molecular weight polyethylene glycols. When aqueous suspensions and/or elixirs are desired for oral administration, the crystalline salt may be combine with various sweetening or flavoring agents, coloring matter or dyes, and, if so desired, emulsifying and/or suspending agents, together with such diluents as water, ethanol, propylene glycol, glycerin and various like combinations thereof. The pharmaceutical compositions of the invention may be formulated such that the crystalline salt is released over a period of time after administration.

Preparing such pharmaceutical compositions of the crystalline salt of the present invention along with a pharmaceutically acceptable excipient and, optionally, an additional active ingredient, may be done by any conventional technique known in the art.

In an embodiment, the crystalline salt present in the pharmaceutical composition is about 0.01% to about 90% by weight relative to the whole composition. A suitable therapeutically effective amount of the crystalline salt will typically range from about 0.01 mg/kg to about 1 g/kg of body weight per day; in another embodiment, from about 1 mg/kg to about 600 mg/kg body weight per day; in another embodiment, from about 1 mg/kg to about 250 mg/kg body weight per day; in another embodiment, from about 10 mg/kg to about 400 mg/kg body weight per day; in another embodiment, from about 10 mg/kg to about 200 mg/kg of body weight per day; in another embodiment, from about 10 mg/kg to about 100 mg/kg of body weight per day; in one embodiment, from about 10 mg/kg to about 25 mg/kg body weight per day; in another embodiment, from about 1 mg/kg to about 10 mg/kg body weight per day; in another embodiment, from about 0.001 mg/kg to about 100 mg/kg of body weight per day; in another embodiment, from about 0.001 mg/kg to about 10 mg/kg of body weight per day; and in another embodiment, from about 0.001 mg/kg to about 1 mg/kg of body weight per day. In a certain embodiment, when a pharmaceutical composition described herein is administered orally, a suitable therapeutically effective amount of the crystalline salt is about 0.01 to about 100 milligrams per kilogram of body weight of recipient per day, preferably about 0.1 to about 50 milligrams per kilogram body weight of recipient per day, more preferably from about 0.1 to about 20 milligrams per kilogram body weight of recipient per day, and even more preferably from about 0.1 to about 10 milligrams per kilogram body weight of recipient per day. The desired dose may be administered once daily, or by several sub-divided doses, e.g., 2 to 5 sub-divided doses, at appropriate intervals through the day, or other appropriate schedule.

The term “pharmaceutically acceptable excipient” as used herein includes, but is not limited to, one of more of the following: polymers, resins, plasticizers, fillers, lubricants, diluents, binders, disintegrants, solvents, co-solvents, surfactants, buffer systems, preservatives, sweetener agents, flavoring agents, pharmaceutical-grade dyes or pigments, chelating agents, viscosity agents, and combinations thereof. Pharmaceutically acceptable excipients can be used in any component in making the dosage form, i.e. core tablet or coating. Flavoring agents and dyes and pigments among those useful herein include but are not limited to those described in Handbook of Pharmaceutical Excipients (4th Ed., Pharmaceutical Press 2003). Suitable co-solvents include, but are not limited to, ethanol, isopropanol, acetone, and combinations thereof. Suitable surfactants include, but are not limited to, polyoxyethylene sorbitan fatty acid esters, polyoxyethylene monoalkyl ethers, sucrose monoesters, simethicone emulsion, sodium lauryl sulfate, Tween 80®, and lanolin esters, ethers, and combinations thereof. Suitable preservatives include, but are not limited to, phenol, alkyl esters of parahydroxybenzoic acid, benzoic acid and the salts thereof, boric acid and the salts thereof, sorbic acid and the salts thereof, chlorbutanol, benzyl alcohol, thimerosal, phenylmercuric acetate and nitrate, nitromersol, benzalkonium chloride, cetylpyridinium chloride, methyl paraben, propyl paraben, and combinations thereof. Suitable fillers include, but are not limited to, starch, lactose, sucrose, maltodextrin, and microcrystalline cellulose. Suitable plasticizers include, but are not limited to, triethyl citrate, polyethylene glycol, propylene glycol, dibutyl phthalate, castor oil, acetylated monoglycerides, triacetin, and combinations thereof. Suitable polymers include, but are not limited to, ethylcellulose, cellulose acetate trimellitate, hydroxypropylmethylcellulose phthalate, cellulose acetate phthalate, polyvinyl acetate phthalate, and Eudragit® L 30-D, Eudragit® L 100-55, Eudragit® F530D and Eudragit® S 100 (Rohm Pharma GmbH and Co. KG, Darmstadt, Germany), Acryl-EZE® and Sureteric® (Colorcon, Inc., West Point, Pa.), and combinations thereof. Suitable lubricants include, but are not limited to, magnesium stearate, stearic acid, talc, and combinations thereof.

The term “additional active ingredient” as used herein includes any agent known in the art to treat, prevent or reduce the symptoms of the condition being treated by the pharmaceutical composition. Such agents, include but are not limited to agents known to treat, prevent or reduce the symptoms of bacterial infections and inflammatory skin disorders. In one embodiment, wherein the pharmaceutical composition of the invention is used in the treatment of a peptic ulcer, the additional active ingredient may include, but not be limited to, a proton pump inhibitor and/or bismuth. Additional agents for treating peptic ulcers are described in Bertram G. Katzung, “Basic and Clinical Pharmacology,” 1064-68 (8^{th} ed., 2001), incorporated by reference herein in its entirety.

In a certain embodiment, the pharmaceutical composition comprises the mono hydrochloride salt of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide and a pharmaceutically acceptable excipient for use in treating a bacterial infection, e.g., methicillin resistant Staphylococcus aureus (MRSA, e.g., MRSA-CA and MRSA-HA), Helicobacter pylori, Chlamydia trachomatis, Chlamydia pneumonia, or conditions associated with such infections (e.g., peptic ulcers and Chlamydia). In another embodiment, the pharmaceutical composition comprises the mono mesylate salt of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide and a pharmaceutically acceptable excipient for use in treating a bacterial infection, e.g., methicillin resistant Staphylococcus aureus (MRSA, e.g., MRSA-CA and MRSA-HA), Helicobacter pylori, Chlamydia trachomatis, Chlamydia pneumonia, or conditions associated with such infections (e.g., peptic ulcers and Chlamydia). In a still further embodiment, the pharmaceutical composition comprises the mono sulfate salt of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide and a pharmaceutically acceptable excipient for use in treating a bacterial infection, e.g., methicillin resistant Staphylococcus aureus (MRSA, e.g., MRSA-CA and MRSA-HA), Helicobacter pylori, Chlamydia trachomatis, Chlamydia pneumonia, or conditions associated with such infections (e.g., peptic ulcers and Chlamydia).

The following examples will illustrate the practice of the present invention in some of the preferred embodiments. Other embodiments within the scope of the claims will be apparent to one skilled in the art.

### EXAMPLES

The following examples illustrate the synthesis of the compounds described herein.

### Synthesis of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide (“the free base”)

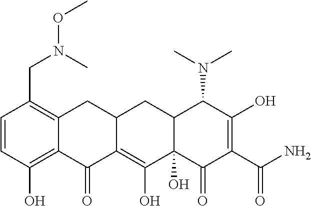 [SMILES: CON(C)Cc1ccc(O)c2c1CC1CC3[C@H](N(C)C)C(O)=C(C(N)=O)C(=O)[C@@]3(O)C(O)=C1C2=O]

A solution of 7-formylsancycline TFA salt (2.23 g) and N,O-dimethylhydroxylamine hydrochloride (780 mg) in N,N-dimethylacetamide (15 mL) was stirred for 10 minutes at room temperature under argon atmosphere. To this solution was added sodium cyanoborohydride (302 mg). The solution was stirred for 5 minutes and monitored by LC-MS. The reaction mixture was poured into diethyl ether, and the resulting precipitates were collected by filtration under vacuum. The crude product was purified by prep-HPLC using a C18 column (linear gradient 10-40% acetonitrile in 20 mM aqueous triethanolamine, pH 7.4). The prep-HPLC fractions were collected, and the organic solvent (acetonitrile) was evaporated under reduced pressure. The resulting aqueous solution was loaded onto a clean PDVB SPE column, washed with distilled water, then with a 0.1 M sodium acetate solution followed by distilled water. The product was eluted with acetonitrile. The eluent was concentrated under reduced pressure, 385 mg was obtained as free base.

### Synthesis of crystalline mono hydrochloride salt of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide (the “Crystalline Mono Hydrochloride Salt”)

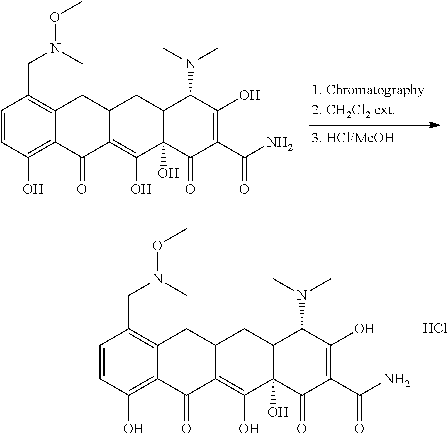

Crude (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide (100 g, app. 35% assay) was purified on preparative column chromatography. The desired fractions (8-10 liters) were combined and the pH was adjusted to 7.0-7.5 using ammonium hydroxide. This aqueous solution was extracted 3 times with dichloromethane (4 liters each time). The dichloromethane layers were combined and concentrated under reduced pressure. The residue was suspended in ethanol (800 ml) and 20 ml water was added. The pH was gradually adjusted to pH 1.6-1.3 using 1.25M hydrochloric acid in methanol and the mixture was stirred for 20-60 minutes at which point the free base was completely dissolved. The solution was concentrated under reduced pressure to 200-250 ml and was seeded with (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide mono HCl crystals (100-200 mg). The stirring was continued for 2-18 hours while the slurry was kept at <5° C. The resulting crystals were filtered, washed with ethanol (50 mL) and dried under reduced pressure to a constant weight. 20 g crystalline (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide mono hydrochloride was isolated in >90% purity and >90% assay.

### Synthesis of crystalline mono mesylate salt of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid (the “Crystalline Mesylate Salt”)

(4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide free base (74 mg) was suspended in ethanol (740 μl) and heated with stirring to 60° C. (bath temperature). Methane sulfonic acid (1.1 eq, 167 μl as 1M solution in THF) was added and most of the solid dissolved. After five minutes, the suspension was cooled to ambient temperature over approximately 1.75 hours (uncontrolled in oil bath). By 53° C., solid had precipitated which was filtered at ambient temperature under reduced pressure. A further portion of ethanol (2000 was added to aid filtration, as the suspension was viscous. The cake was washed with n-hexane (4000 and air dried on filter for approximately 30 minutes to yield 59 mg (67% yield) of yellow solid.

### Synthesis of crystalline mono sulfate salt of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid (the “Crystalline Sulfate Salt”)

(4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide free base (86 mg) was suspended in ethanol (500 μl) and heated with stirring to 63° C. (bath temperature) at which temperature most of the free base had dissolved. Sulfuric acid (1.1 eq, 194 μl as 1M solution in water) was added and all of the solid dissolved. The solution was cooled to ambient temperature over approximately 1.75 hours (uncontrolled in oil bath) at which temperature no solid had precipitated. Methyl t-butyl ether (MtBE) was added as an antisolvent (4×50 μl). Each addition caused a cloud point, but the solid re-dissolved on stirring. The solution was stirred with a stopper for approximately 3 hours after which time solid precipitated. The solid was filtered under reduced pressure and washed with MtBE (3×200 μl) and air dried on filter for approximately 45 minutes to yield 93 mg (90% yield) of yellow solid.

### Synthesis of amorphous bis hydrochloride salt of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide

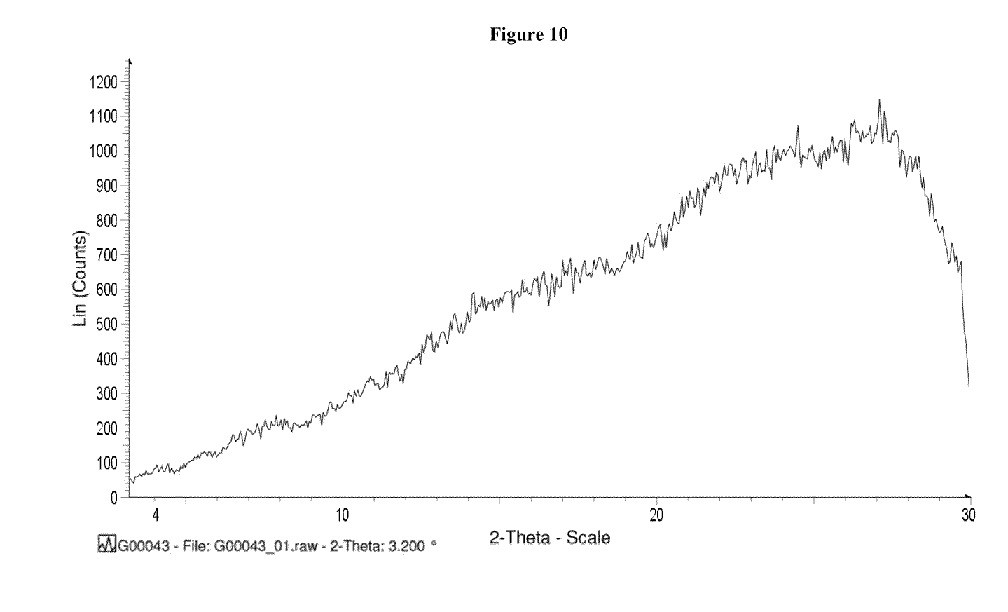

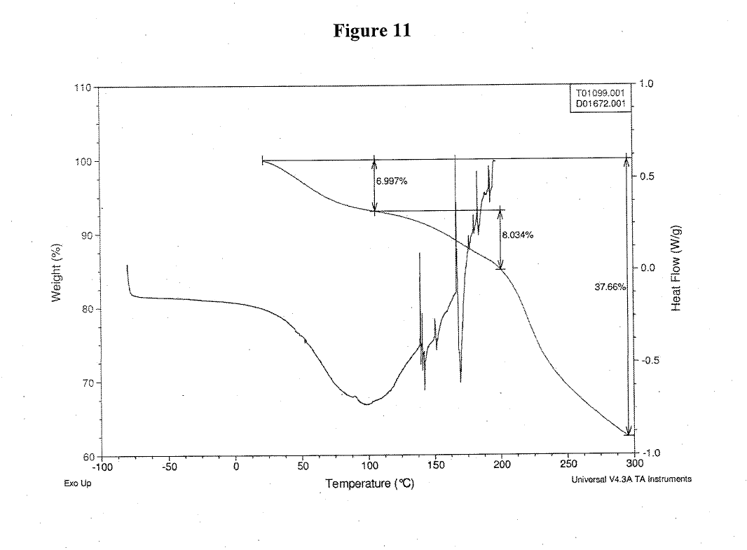

(4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide free base (1 g) was suspended in methanol (50 mL). The freebase was converted to the hydrochloride salt by adding an excess of methanolic HCl followed by under reduced pressure evaporation to give 1.1 g yellow solid: MS (Mz+1=488). 1H NMR (300 MHz, CD3OD) δ 7.46 (d, 1H, J=8.6 Hz), 6.81 (d, 1H, J=8.6 Hz), 4.09 (d, 1H, J=1.0 Hz), 3.79 (d, 1H, J=13.1 Hz), 3.73 (d, 1H, J=13.1 Hz), 3.36 (m, 1H), 3.27 (s, 3H), 3.08-2.95 (8H), 2.61 (s, 3H), 2.38 (t, 1H, J=14.8), 2.22 (m, 1H), 1.64 (m, 1H). An XRPD pattern is shown in FIG. 10 and a TGA and DSC curve overlaid are shown in FIG. 11.

### Synthesis of amorphous mono hydrochloride salt of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide

A sample of Crystalline Mono Hydrochloride Salt (2.09 g) was dissolved in water (250 ml, 120 vols), filtered and frozen in a −78° C. bath. Water was removed from the solidified sample using a lyophilizer for 110 hours to yield the amorphous mono hydrochloride salt as a fluffy yellow solid, that was confirmed to be amorphous by XRPD analysis.

### Testing

### Antimicrobial Activity

Antimicrobial activity of the Crystalline Mono Hydrochloride Salt was assessed according to anti-anaerobic activity and mechanism of action studies as detailed herein.

For the studies below, samples were prepared with bis hydrochloride salt, and the data is expressed based on the free base (“active”). The overall anti-anaerobic microbial activity of the Crystalline Mono Hydrochloride Salt can be seen via in vitro study of the active against 37 representative strains of anaerobic bacteria and the results compiled in Table 1. The active demonstrated relatively potent activity (i.e., minimum inhibitory concentration (MIC) of 4 μg/mL or less) against many species of Gram-positive bacteria, including P. acnes. Overall, the activity of the active was similar to that of tetracycline and doxycycline but less than that of minocycline. Organisms with high MIC values for the active (MIC >16 μg/mL) included C. perfringens and S. constellatus.

MIC values for the Gram-negative anaerobes are shown in Table 1. The tetracycline-resistant strains were cross-resistant to the active. The active and the other tetracyclines demonstrated potent activity against E. corrodens and Fusobacterium spp., moderate activity against P. melaminogenica (1 of 2 strains) and V. parvula, and poor activity against P. asaccharolytica.

TABLE 1
Summary of in vitro MIC testing of the Active Against
Anaerobic Gram-Positive and Gram-Negative Bacteria.

| | | The | | | |
|---|---|---|---|---|---|
| | | Active | TET | DOX | MIN |
| Organism/ | ATCC | MIC | MIC | MIC | MIC |
| Micromyx No. | No. | (μg/mL) | (μg/mL) | (μg/mL) | (μg/mL) |
| Bifidobacterium bifidum | 15696 | 1 | 1 | 0.5 | 0.25 |
| 3965 | | | | | |
| Bifidobacterium brevi | 15698 | 1 | 1 | 0.5 | 0.25 |
| 3967 | | | | | |
| Bifidobacterium infantis | 15702 | 0.5 | 1 | 0.5 | 0.25 |
| 3966 | | | | | |
| Bifidobacterium longum | 15707 | 4 | 2 | 1 | 1 |
| 3968 | | | | | |
| Clostridium perfringens | — | 16 | >16 | 16 | 16 |
| 3414 | | | | | |
| Clostridium perfringens | — | 16 | >16 | 16 | 8 |
| 3518 | | | | | |
| Clostridium difficile | — | 0.12 | 0.5 | 0.06 | 0.03 |
| 3579 | | | | | |
| Clostridium difficile | — | 0.12 | 0.5 | 0.06 | 0.03 |
| 3584 | | | | | |
| Lactobacillus | — | 4 | 2 | 2 | 0.5 |
| acidophilus 0681 | | | | | |
| Lactobacillus casei 1722 | 393 | 2 | 2 | 2 | 0.5 |
| Lactobacillus plantarum | 39268 | 2 | 2 | 2 | 0.5 |
| 2791 | | | | | |
| Peptostreptococcus | — | 2 | 8 | 2 | 1 |
| anaerobius 3526 | | | | | |
| Peptostreptococcus | — | 4 | 16 | 4 | 2 |
| anaerobius 3531 | | | | | |
| Peptostreptococcus | — | 0.25 | 0.25 | 0.12 | 0.06 |
| micros 3432 | | | | | |
| Peptostreptococcus | — | 1 | 1 | 0.5 | 0.25 |
| micros 3545 | | | | | |
| Propionibacterium acnes | — | 0.25 | 0.25 | 0.12 | 0.06 |
| 1713 | | | | | |
| Propionibacterium acnes | 11829 | 1 | 1 | 0.5 | 0.5 |
| 1286 | | | | | |
| Streptococcus | 27823 | 32 | >16 | 16 | 16 |
| constellatus 1202 | | | | | |
| Streptococcus | 27335 | 1 | 2 | 0.5 | 0.25 |
| intermedius 1203 | | | | | |
| Bacteroides fragilis 3374 | — | 0.12 | 0.5 | 0.12 | 0.03 |
| Bacteroides fragilis 3479 | — | 16 | >16 | 16 | 8 |
| Bacteroides ovatus 3503 | — | 8 | >16 | 8 | 4 |
| Bacteroides ovatus 3508 | — | 0.25 | 0.5 | 0.12 | 0.03 |
| Bacteroides | — | 0.25 | 1 | 0.25 | 0.03 |
| thetaiotaomicron 3399 | | | | | |
| Bacteroides | — | 16 | >16 | 16 | 8 |
| thetaiotaomicron 3496 | | | | | |
| Bacteroides vulgatus | — | 16 | >16 | 8 | 8 |
| 3389 | | | | | |
| Bacteroides vulgatus | — | 16 | >16 | 8 | 8 |
| 3494 | | | | | |
| Eikenella corrodens | 43278 | 1 | 0.5 | 0.12 | 0.03 |
| 1206 | | | | | |
| Fusobacterium | 25286 | 0.25 | 0.5 | 0.5 | 0.06 |
| necrophorum 3963 | | | | | |
| Fusobacterium | 25586 | 0.25 | 0.5 | 0.5 | 0.06 |
| nucleatum 3962 | | | | | |
| Porphyromonas | — | 16 | >16 | 4 | 8 |
| asaccharolytica 3552 | | | | | |
| Porphyromonas | — | 8 | 16 | 2 | 4 |
| asaccharolytica 3557 | | | | | |
| Prevotella | — | 32 | >16 | 16 | 16 |
| melaninogenica 3437 | | | | | |
| Prevotella | — | 4 | 8 | 1 | 1 |
| melaninogenica 3443 | | | | | |
| Prevotella spp. 3564 | — | 4 | 1 | 1 | 0.25 |
| Prevotella spp. 3568 | — | 2 | 4 | 1 | 0.25 |
| Veillonella parvula 1272 | 17745 | 4 | 1 | 1 | 0.5 |

“TET” is tetracycline;
“DOX” is doxycycline;
“MIN” is minocycline;
“ATCC” is American Type Culture Collection.

### Antibacterial Spectrum of Activity

An assessment of the antibacterial spectrum of activity of the active was determined in several studies by in vitro MIC determination for a variety of Gram-positive and Gram-negative aerobic and anaerobic organisms. The results of these assays (summarized in Table 2) indicate that the active demonstrates activity against propionibacteria and other Gram-positive organisms with a narrower spectrum of activity than clinically-used tetracyclines. Strains resistant to tetracycline are cross-resistant to the active. The activity for each organism group is discussed in the text that follows the table.

TABLE 2
Summary of In Vitro MIC testing against propionibacteria and
aerobic Gram-positive and Gram-negative organisms

| | | MIC | | |
|---|---|---|---|---|
| Organism [Type] (No. | | Range | MIC₅₀ | MIC₉₀ |
| isolates) | Compound | (μg/mL) | (μg/mL) | (μg/mL) |
| Propionibacteria | | | | |
| P. acnes | The Active | 0.25-4 | 0.5 | 2 |
| [tetS] | Tetracycline | 0.5-4 | 0.5 | 4 |
| (13) | Doxycycline | 0.25-1 | 0.25 | 1 |
| | Minocycline | ≦0.06->8 | 0.125 | 1 |
| P. acnes | The Active | >8->8 | | |
| [tetR] | Tetracycline | >8->8 | | |
| (2) | Doxycycline | 8-8 | | |
| | Minocycline | 2-2 | | |
| P. acnes | The Active | 0.5-16 | 0.5 | 4 |
| [clinical isolates] | Tetracycline | 0.5-32 | 1 | 2 |
| (55) | Doxycycline | 0.25-16 | 0.5 | 2 |
| | Minocycline | 0.125-8 | 0.25 | 1 |
| | Clindamycin | ≦0.06-64 | ≦0.06 | 4 |
| | Erythromycin | ≦0.06->128 | ≦0.06 | >128 |
| P. acnes | The Active | 0.25-1 | | |
| [tetS] | Tetracycline | 0.25-1 | | |
| (2) | Doxycycline | 0.12-0.5 | | |
| | Minocycline | 0.06-0.5 | | |
| | Clindamycin | 0.06-0.25 | | |
| | Metronidazole | >32->32 | | |
| | Penicillin | 0.03-0.5 | | |
| | Vancomycin | 0.5-0.5 | | |
| P. granulosum | The Active | 1-1 | | |
| [clinical isolates] | Tetracycline | 1-2 | | |
| (3) | Doxycycline | 0.5-1 | | |
| | Minocycline | 0.25-0.5 | | |
| | Clindamycin | ≦0.06-≦0.06 | | |
| | Erythromycin | ≦0.06-≦0.06 | | |
| P. avidum | The Active | 1-4 | | |
| [clinical isolates] | Tetracycline | 1-8 | | |
| (4) | Doxycycline | 0.5-4 | | |
| | Minocycline | 0.25-2 | | |
| | Clindamycin | ≦0.06-0.5 | | |
| | Erythromycin | 0.125-0.125 | | |
| Gram-positive aerobic | | | | |
| bacteria | | | | |
| S. aureus | The Active | ≦0.06-0.25 | 0.125 | 0.25 |
| [tetS] | Tetracycline | ≦0.06-0.25 | 0.25 | 0.25 |
| (20) | Doxycycline | ≦0.06-0.25 | ≦0.06 | 0.25 |
| | Minocycline | 0.125-0.5 | 0.25 | 0.5 |
| S. aureus | The Active | 0.125-32 | 4 | 16 |
| [tetR] | Tetracycline | 2-64 | 64 | 64 |
| (10) | Doxycycline | 1-16 | 4 | 16 |
| | Minocycline | 0.25-16 | 0.5 | 8 |
| S. aureus | The Active | 0.25-16 | 0.5 | 0.5 |
| [MSSA] | Tetracycline | 0.25->32 | 0.25 | 0.5 |
| (32) | Doxycycline | 0.12-8 | 0.12 | 0.25 |
| | Minocycline | 0.06-8 | 0.12 | 0.12 |
| | Erythromycin | 0.25->32 | 0.5 | >32 |
| | Clindamycin | 0.12->32 | 0.25 | >32 |
| | Oxacillin | 0.12-1 | 0.5 | 1 |
| | Vancomycin | 0.5-1 | 1 | 1 |
| S. aureus | The Active | 0.25-4 | 0.25 | 0.5 |
| [MRSA] | Tetracycline | 0.25-2 | 0.25 | 0.5 |
| (31) | Doxycycline | 0.12-2 | 0.12 | 0.25 |
| | Minocycline | 0.06-0.5 | 0.06 | 0.12 |
| | Erythromycin | 0.5->32 | >32 | >32 |
| | Clindamycin | 0.12->32 | 0.25 | >32 |
| | Oxacillin | 4->32 | >32 | >32 |
| | Vancomycin | 0.5-2 | 1 | 1 |
| S. epidermidis | The Active | 0.12-2 | 0.25 | 2 |
| [MSSE] | Tetracycline | 0.12-2 | 0.25 | 2 |
| (31) | Doxycycline | 0.06-1 | 0.12 | 1 |
| | Minocycline | 0.06-0.25 | 0.06 | 0.25 |
| | Erythromycin | 0.12->32 | 0.25 | >32 |
| | Clindamycin | ≦0.03->32 | 0.12 | >32 |
| | Oxacillin | 0.06-0.25 | 0.12 | 0.25 |
| | Vancomycin | 1-2 | 2 | 2 |
| S. epidermidis | The Active | 0.25-2 | 0.5 | 2 |
| [MRSE] | Tetracycline | 0.25->32 | 1 | 2 |
| (32) | Doxycycline | 0.12-8 | 0.5 | 1 |
| | Minocycline | 0.06-0.5 | 0.12 | 0.25 |
| | Erythromycin | 0.12->32 | >32 | >32 |
| | Clindamycin | 0.06->32 | >32 | >32 |
| | Oxacillin | 0.5->32 | 32 | >32 |
| | Vancomycin | 1-2 | 2 | 2 |
| S. pneumoniae | The Active | ≦0.06-0.125 | | |
| [tetS] | Tetracycline | ≦0.06-0.25 | | |
| (5) | Doxycycline | ≦0.06-0.125 | | |
| | Minocycline | 0.25-0.25 | | |
| S. pneumoniae | The Active | 4-32 | | |
| [tetR] | Tetracycline | 32-64 | | |
| (5) | Doxycycline | 4-4 | | |
| | Minocycline | 8-16 | | |
| S. pneumoniae | The Active | ≦0.03-32 | 0.12 | 0.25 |
| [PSSP] | Tetracycline | 0.06->32 | 0.12 | 0.25 |
| (32) | Doxycycline | 0.03->16 | 0.06 | 0.12 |
| | Minocycline | ≦0.015->16 | 0.06 | 0.12 |
| | Erythromycin | ≦0.015->16 | 0.03 | 2 |
| | Clindamycin | ≦0.015->16 | 0.03 | 0.06 |
| | Penicillin | ≦0.015-0.12 | ≦0.015 | 0.06 |
| | Vancomycin | 0.06-0.25 | 0.25 | 0.25 |
| S. pyogenes | The Active | 0.12-16 | 0.12 | 8 |
| (32) | Tetracycline | 0.12-32 | 0.12 | 32 |
| | Doxycycline | 0.06-8 | 0.12 | 4 |
| | Minocycline | 0.03-8 | 0.06 | 8 |
| | Erythromycin | 0.03->16 | 0.06 | 0.06 |
| | Clindamycin | 0.03->16 | 0.03 | 0.06 |
| | Penicillin | ≦0.015-0.25 | ≦0.015 | ≦0.015 |
| | Vancomycin | 0.25-0.5 | 0.25 | 0.25 |
| S. pyogenes | The Active | ≦0.06-0.25 | | |
| [tetS] | Tetracycline | ≦0.06-0.125 | | |
| (5) | Doxycycline | ≦0.06-0.125 | | |
| | Minocycline | 0.25-0.5 | | |
| S. pyogenes | The Active | 4-16 | | |
| [tetR] | Tetracycline | 32-64 | | |
| (5) | Doxycycline | 4-8 | | |
| | Minocycline | 4-8 | | |
| S. agalactiae | The Active | 0.12-32 | 16 | 16 |
| (31) | Tetracycline | 0.12->32 | 32 | >32 |
| | Doxycycline | 0.06-16 | 8 | 16 |
| | Minocycline | 0.03-16 | 16 | 16 |
| | Erythromycin | 0.03->16 | 0.06 | >16 |
| | Clindamycin | 0.03->16 | 0.06 | >16 |
| | Penicillin | ≦0.015-2 | 0.03 | 1 |
| | Vancomycin | 0.25-2 | 0.5 | 0.5 |
| S. agalactiae | The Active | 0.125-0.25 | | |
| [tetS] | Tetracycline | 0.25-0.25 | | |
| (3) | Doxycycline | 0.25-0.25 | | |
| | Minocycline | 0.5-0.5 | | |
| S. agalactiae | The Active | 16-32 | | |
| [tetR] | Tetracycline | 16-64 | | |
| (7) | Doxycycline | 8-16 | | |
| | Minocycline | 8-16 | | |
| S. haemolyticus | The Active | 0.12-2 | 0.12 | 2 |
| (33) | Tetracycline | 0.12->32 | 1 | >32 |
| | Doxycycline | 0.06-16 | 0.5 | 16 |
| | Minocycline | ≦0.03-0.5 | 0.06 | 0.5 |
| | Erythromycin | 0.12->32 | >32 | >32 |
| | Clindamycin | 0.06->32 | 0.12 | 1 |
| | Oxacillin | 0.06->32 | 0.25 | >32 |
| | Vancomycin | 0.5-2 | 1 | 1 |
| Streptococcus | The Active | 0.12-16 | 0.25 | 16 |
| spp. | Tetracycline | 0.12->32 | 0.25 | 32 |
| [Group C] | Doxycycline | 0.06-16 | 0.12 | 8 |
| (30) | Minocycline | 0.03-8 | 0.06 | 8 |
| | Erythromycin | ≦0.015->16 | 0.06 | 4 |
| | Clindamycin | ≦0.015->16 | 0.06 | 0.12 |
| | Penicillin | ≦0.015-0.03 | ≦0.015 | ≦0.015 |
| | Vancomycin | 0.25-1 | 0.25 | 0.5 |
| E. faecalis | The Active | ≦0.06-≦0.06 | | |
| [tetS] | Tetracycline | 0.25-0.5 | | |
| (4) | Doxycycline | ≦0.06-0.125 | | |
| | Minocycline | 0.25-0.5 | | |
| E. faecalis | The Active | 8-32 | | |
| [tetR] | Tetracycline | 32-64 | | |
| (6) | Doxycycline | 2-16 | | |
| | Minocycline | 4-16 | | |
| E. faecalis | The Active | 0.25-32 | 32 | 32 |
| [VSE] | Tetracycline | 0.25->64 | 32 | 64 |
| (31) | Doxycycline | 0.12-16 | 8 | 8 |
| | Minocycline | 0.06-16 | 8 | 16 |
| | Erythromycin | 0.25->32 | >32 | >32 |
| | Clindamycin | 4->32 | >32 | >32 |
| | Ampicillin | 0.5-8 | 1 | 1 |
| | Vancomycin | 0.5-4 | 1 | 2 |
| E. faecium | The Active | ≦0.06-≦0.06 | | |
| [tetS] | Tetracycline | 0.125-0.25 | | |
| (4) | Doxycycline | ≦0.06-≦0.06 | | |
| | Minocycline | 0.25-0.5 | | |
| E. faecium | The Active | 8-32 | | |
| [tetR] | Tetracycline | 32-64 | | |
| (6) | Doxycycline | 4-16 | | |
| | Minocycline | 2-32 | | |
| E. faecium | The Active | 0.12-32 | 0.5 | 32 |
| [VSE] | Tetracycline | 0.12->64 | 1 | >64 |
| (32) | Doxycycline | 0.06-32 | 0.5 | 16 |
| | Minocycline | ≦0.03-16 | 0.12 | 16 |
| | Erythromycin | 0.06->32 | >32 | >32 |
| | Clindamycin | 0.12->32 | >32 | >32 |
| | Ampicillin | 0.12->64 | 64 | >64 |
| | Vancomycin | 0.25-2 | 1 | 1 |
| E. faecium | The Active | 0.12-32 | 2 | 32 |
| [VRE] | Tetracycline | 0.12->64 | 2 | >64 |
| (30) | Doxycycline | 0.06-16 | 1 | 8 |
| | Minocycline | ≦0.03-16 | 0.25 | 16 |
| | Erythromycin | 0.12->32 | >32 | >32 |
| | Clindamycin | 0.06->32 | >32 | >32 |
| | Ampicillin | 8->64 | >64 | >64 |
| | Vancomycin | >64 | >64 | >64 |
| Gram-negative aerobic | | | | |
| bacteria | | | | |
| E. coli | The Active | 4-32 | | |
| [tetS] | Tetracycline | 1-4 | | |
| (7) | Doxycycline | 0.5-4 | | |
| | Minocycline | 0.5-4 | | |
| E. coli | The Active | >64->64 | | |
| [tetR] | Tetracycline | >64->64 | | |
| (3) | Doxycycline | 64-64 | | |
| | Minocycline | 8-16 | | |
| E. coli | The Active | 2->64 | 16 | >64 |
| (33) | Tetracycline | 1->64 | 2 | >64 |
| | Doxycycline | 0.5->32 | 2 | 32 |
| | Minocycline | 0.25->32 | 1 | 8 |
| | Ampicillin | 1->64 | >64 | >64 |
| | Ciprofloxacin | 0.008->2 | 0.015 | >2 |
| | Cephalothin | 2->64 | 32 | >64 |
| | Tmp/Sxt | ≦0.06/1.19->64/1216 | 0.25/4.75 | >64/1216 |
| K. pneumoniae | The Active | 16-64 | | |
| [tetS] | Tetracycline | 0.5-4 | | |
| (7) | Doxycycline | 0.5-8 | | |
| | Minocycline | 1-16 | | |
| K. pneumoniae | The Active | >64->64 | | |
| [tetR] | Tetracycline | 8->64 | | |
| (5) | Doxycycline | 16-64 | | |
| | Minocycline | 16->64 | | |
| K. pneumoniae | The Active | 16->64 | >64 | >64 |
| (31) | Tetracycline | 1->64 | 8 | >64 |
| | Doxycycline | 1->32 | 8 | >32 |
| | Minocycline | 1->32 | 4 | >32 |
| | Ampicillin | >64 | >64 | >64 |
| | Ciprofloxacin | 0.03->2 | >2 | >2 |
| | Cephalothin | >64 | >64 | >64 |
| | Tmp/Sxt | 0.12/2.38->64/1216 | >64/1216 | >64/1216 |
| E. cloacae | The Active | 0.25->64 | 32 | >64 |
| (30) | Tetracycline | 0.5->64 | 2 | >64 |
| | Doxycycline | 0.06->32 | 2 | 32 |
| | Minocycline | ≦0.03->32 | 1 | 16 |
| | Ampicillin | 4->64 | 64 | >64 |
| | Ciprofloxacin | 0.008->2 | 0.25 | >2 |
| | Cephalothin | 2->64 | >64 | >64 |
| | Tmp/Sxt | ≦0.06/1.19->64/1216 | 0.25/4.75 | >64/1216 |
| P. mirabilis | The Active | >64 | >64 | >64 |
| (30) | Tetracycline | 16->64 | 32 | 64 |
| | Doxycycline | 32->32 | >32 | >32 |
| | Minocycline | 8->32 | 16 | >32 |
| | Ampicillin | 0.5->64 | 4 | >64 |
| | Ciprofloxacin | 0.015->2 | >2 | >2 |
| | Cephalothin | 2->64 | 8 | >64 |
| | Tmp/Sxt | ≦0.06/1.19->64/1216 | 2/38 | >64/1216 |
| P. aeruginosa | The Active | 32->64 | >64 | >64 |
| (11) | Tetracycline | 4->64 | 64 | 64 |
| | Doxycycline | 4->32 | >32 | >32 |
| | Minocycline | 8->32 | >32 | >32 |
| | Ampicillin | >64 | >64 | >64 |
| | Ciprofloxacin | 0.12->2 | >2 | >2 |
| | Cephalothin | >64 | >64 | >64 |
| | Tmp/Sxt | 2/38->64/1216 | 16/304 | >64/1216 |
| Salmonella | The Active | 8->64 | 16 | >64 |
| Spp. | Tetracycline | 1->64 | 2 | >64 |
| (35) | Doxycycline | 2->32 | 2 | 32 |
| | Minocycline | 1->32 | 2 | 8 |
| | Ampicillin | 0.5->64 | 1 | >64 |
| | Ciprofloxacin | 0.015-0.25 | 0.015 | 0.03 |
| | Cephalothin | 1->64 | 2 | 16 |
| | Tmp/Sxt | ≦0.06/1.19->64/1216 | ≦0.06/1.19->64/1216 | 0.12/2.38 |

Abbreviations used in Table 2:
tetS, tetracycline sensitive;
tetR, tetracycline resistant;
VSE, vancomycin-susceptible Enterococcus;
VRE, vancomycin-resistant Enterococcus;
MSSA, methicillin-susceptible Staphylococcus aureus;
MRSA, methicillin-resistant Staphylococcus aureus;
MSSE, methicillin-susceptible Staphylococcus epidermidis;
MRSE, methicillin-resistant Staphylococcus epidermidis;
PSSP, penicillin-susceptible Streptococcus pneumoniae;
MIC, minimum inhibitory concentration;
MIC₅₀, MIC at which 50% of isolates are inhibited;
MIC₉₀, MIC at which 90% of the isolates are inhibited.

Activity Against Chlamydia trachomatis

For the studies below, samples were prepared with crystalline mono hydrochloride salt, and the data is expressed based on the free base (“the active”). In vitro activity of the compound against Chlamydia trachomatis was compared with that of azitromycin, levofloxacin, and doxycycline. In vitro susceptibility testing was performed in HEp-2 cells. The MIC for the active at which 90% of the strains of C. trachomatis were inhibited (MIC₉₀) was 0.125 μg/mL (range 0.06-0.125 μg/mL). The minimum bactericidal concentration at which 90% of the strains were inhibited (MBC₉₀) was also 0.125 μg/mL. These data were similar to data obtained from the comparators (MIC₉₀ for azitromycin, levofloxacin, and doxycycline were 0.015, 0.25, and 0.125 μg/mL, respectively). The MICs were very consistent from isolate to isolate, regardless of the geographical distribution of the isolates tested.

Activity Against Helicobacter pylori (H. pylori)

For the studies below, samples were prepared with crystalline mono hydrochloride salt, and the data is expressed based on the free base (“the active”). In vitro activity of the compound against various strains of H. pylori was compared with that of amoxicillin, tetracycline, and metronidazole (see Table 3). The active demonstrated consisted activity for the strains tested, inhibiting all test strains at 8 μg/mL or less, with 12 out of 13 strains inhibited by 1-2 μg/mL. For the clinical isolates, MIC₉₀ value was 2 μg/mL. The active was generally less active than the three comparator agents; however, the level of activity is significant in view of drug levels that may be achieved in the upper gastrointestinal tract following oral dosing.

TABLE 3
MIC Values for the Active and Comparator Agents against
Multiple Isolates of H. pylori

| | | | MIC, μg/mL | | | |
|---|---|---|---|---|---|---|
| | Cul- | | (CLSI Quality Control Range)² | | | |
| | ture | ATCC¹ | The | | | |
| | No. | Number | active | AMX⁵ | TET⁶ | MTZ⁷ |
| Reference | 3718 | 43504 | 2 | 0.03 | 1 | 64 (64-256) |
| Strains | | | | (0.015-0.12) | (0.12-1) | |
| | 3719 | 700824 | 2 | 0.015 | 0.5 | 1 |
| | 3720 | BAA- | 1 | 0.06 | 0.5 | 1 |
| | | 945 | | | | |
| Clinical | 4368 | — | 2 | 0.06 | 1 | 0.25 |
| Isolates | 4369 | — | 8 | 0.12 | 2 | 0.5 |
| | 4370 | — | 2 | 0.12 | 0.5 | 0.12 |
| | 4371 | — | 2 | 0.12 | 0.5 | 1 |
| | 4372 | — | 1 | 0.015 | 1 | 0.25 |
| | 4374 | — | 2 | 0.015 | 1 | 0.12 |
| | 4375 | — | 2 | 0.12 | 0.5 | 0.12 |
| | 4377 | — | 1 | 0.015 | 0.25 | 1 |
| | 4503 | — | 2 | 0.03 | 0.5 | ≦0.03 |
| | 4504 | — | 1 | 0.015 | 0.5 | ≦0.03 |

| | | | | | |
|---|---|---|---|---|---|
| Clinical Isolate | MIC | 1-8 | 0.015-0.12 | 0.25-2 | ≦0.03-64 |
| Summary | Range | | | | |
| | MIC₅₀³ | 2 | 0.03 | 0.5 | 0.12 |
| | MIC₉₀⁴ | 2 | 0.12 | 1 | 1 |

¹American Tissue Culture Collection Number
²Clinical and Laboratory Standards Institute quality control range
³MIC for 50% of clinical test isolates
⁴MIC for 90% of clinical test isolates
⁵AMX—amoxicillin
⁶TET—tetracycline
⁷MTZ—metronidazole

Activity Against Methicillin-Resistant Staphylococcus aureus (MRSA)

For the studies below, samples were prepared with crystalline mono hydrochloride salt, and the data is expressed based on the free base (“the active”). In vitro activity of the compound against various strains of either methicillin-sensitive S. aureus (MSSA), hospital-associated methicillin-resistant S. aureus (MRSA-HA), or community-acquired methicillin-resistant S. aureus (MRSA-CA) was compared with that of doxycycline, oxacillin, clindamycin, linezolid, and a combination of trimethoprim/sulfamethoxazole (TMP/SXT), oral antibacterial agents commonly used for the treatment of MRSA. The MIC results are summarized in Table 4. The active was very active against MSSA with MIC range of 0.25-16 μg/mL, MIC₅₀ of 0.25 μg/mL, and MIC₉₀ of 1 μg/mL; values similar to those of doxycycline and oxacillin. A single strain of doxycycline-resistant MSSA (DOX MIC of 8 μg/mL) also had an elevated MIC (16 μg/mL) for the active. The active was less active than TMP/SXT, but more active than clindamycin or linezolid against MSSA. All of the test organisms were susceptible to linezolid.

The active demonstrated potent activity against MRSA-HA, equivalent to that of doxycycline, with MIC₅₀ and MIC₉₀ values of 0.25 μg/mL and 0.5 μg/mL, respectively. The active was less active than TMP/SXT and more active than the rest of the comparator agents. All of the organisms were susceptible to linezolid.

Finally, the active demonstrated potent activity against MRSA-CA with an MIC₉₀ value of 0.5 μg/mL that was similar to MIC₉₀ for doxycycline and clindamycin. A single strain of doxycycline-resistant MRSA-CA (DOX MIC of 8 μg/mL) also had an elevated MIC (8 μg/mL) for the active. The active was less active than TMP/SXT, but more active than oxacillin and linezolid. All of the test organisms were susceptible to linezolid.

TABLE 4
MIC Values for the Active and Comparator Agents against
Multiple Isolates of MSSA, MRSA-HA, and MRSA-CA

| Organism | | MIC | | |
|---|---|---|---|---|
| (No. | | Range | MIC₅₀ | MIC₉₀ |
| Isolates) | Drug | (μg/mL) | (μg/mL) | (μg/mL) |
| MSSA | The Active | 0.25-16 | 0.25 | 1.0 |
| (34) | Doxycycline | 0.12-8 | 0.25 | 0.5 |
| | Oxacillin | 0.25-2 | 0.25 | 0.5 |
| | Clindamycin | 0.06->32 | 0.12 | >32 |
| | Linezolid | 2-4 | 2 | 4 |
| MRSA-HA | The Active | 0.25-0.5 | 0.25 | 0.5 |
| (33) | Doxycycline | 0.12-1 | 0.25 | 0.5 |
| | Oxacillin | 8->32 | 32 | >32 |
| | Clindamycin | 0.06->32 | 2 | >32 |
| | Linezolid | 2-4 | 2 | 2 |
| MRSA-CA | The Active | 0.12-8 | 0.25 | 0.5 |
| (33) | Doxycycline | 0.12-8 | 0.12 | 2 |
| | Oxacillin | 16->32 | 32 | >32 |
| | Clindamycin | 0.06->32 | 0.12 | 0.25 |
| | Linezolid | 2-4 | 2 | 4 |
| MSSA | TMP/SXT | 0.03/0.6-0.12/2.5 | 0.06/1.2 | 0.06/1.2 |
| (34) | | | | |
| MRSA-HA | | 0.03/0.6->16/304 | 0.06/1.2 | 0.12/2.5 |
| (33) | | | | |
| MRSA-CA | | ≦0.015/0.3-0.25/5 | 0.06/1.2 | 0.12/2.5 |
| (33) | | | | |

Activity Against Chlamydia trachomatis and Chlamydia pneumoniae

For this study, samples were prepared with crystalline mono hydrochloride salt, and the data is expressed based on the free base (“the active”). In vitro activity of the active against 10 isolates of Chlamydia trachomatis (also referred to as C. trachomatis) and Chlamydia pneumoniae (also referred to as C. pneumoniae) was studied and compared to the activities of 3 comparators: levofloxacin, azithromycin, and doxycycline.

The following materials were used in the study:
1. Chlamydia isolates
   Isolates of C. trachomatis included standard isolates from the ATCC®: D-UW-57Cx (VR-878), E-BOUR (VR-348B), F-IC-CAL3 (VR-346), H-UW-43Cx (VR-879), I-UW-12Ur (VR-880), J-UW-36Cx (VR-886), L2-434 (VR-902B) and clinical isolates N18 (cervical), N19 (cervical), 7015 (infant eye).
   Isolates of C. pneumoniae tested included 5 isolates from standard isolates from the ATCC: TW 183, AR 39 (53592), CM-1 (VR-1360), T 2040, and 6 isolates from bronchoalveolar lavage specimens from patients with human immunodeficiency virus infection and pneumonia from the United States (BAL15, BAL16, BAL 18, BAL 19, BAL 37, BAL 62).
2. HEp2 cells: ATCC® Manassas, Va. ATCC® Number: CCL-23™ Lot #58978772
3. Antibiotics
   a. The active
   b. 3 comparators manufactured by Sigma-Aldrich®
      i. Levofloxacin Catalog Number 28266-1G-F Lot # BCBF7004V
      ii. Doxycycline Catalog Number D9891-1G Lot # BCBD5187V
      iii. Azithromycin Catalog Number 75199-25MG-F Lot # E446421/1V
4. IMDM with L-glutamine and Phenol Red: Gibco® Catalog Number 12440-061 Lot #1153614
5. FBS: Gibco® Catalog Number 10438-026 Lot #1140649
6. Cycloheximide: Sigma-Aldrich® Catalog Number C4859 Lot #090M4009
7. PBS: Gibco® Catalog Number 10010-072 Gibco Lot #1144930
8. Pathfinder® Chlamydia Culture Confirmation System: Biorad® Catalog Number 30701 Lot #109515

The active, azithromycin, levofloxacin, and doxycycline were provided as powders and solubilized according to the manufacturers' instructions. Stock solutions of 1280 μg/ml were made and frozen at −80° C. Aliquots of the stock drug suspensions were diluted each time the assay was run. Chlamydia isolates were expanded to concentrations of 10⁷ to 10⁸ inclusion-forming units (IFU) per ml by serial passage in tissue culture using antibiotic-free media. Isolates were purified by centrifugation at 500 rpm to bring down the cell debris. The chlamydia containing supernatant was pelleted at 17,000×g for 1 hour. The pellet containing the chlamydia was then resuspended in Sucrose Phosphate Glutamate Medium (SPG) and centrifuged through a discontinuous renografin gradient. SPG is composed of sucrose (250 mM), glutamic acid (5 mM), sodium phosphate (10 mM), and 20% Fetal Calf Serum, and has a pH of 7.4. The chlamydial elementary body (EB) containing band was then washed ×3 and resuspended in SPG. The EB suspension is titered in HEp-2 cells.

Susceptibility testing of C. pneumoniae and C. trachomatis was performed in cell culture using HEp-2 cells grown in 96-well microtiter plates. Each well was inoculated with 0.2 ml of the test strain diluted to yield 10⁴ inclusion-forming units per ml; the plates were centrifuged at 1,700×g for 1 h and incubated at 35° C. for 1 h. Wells were then aspirated and overlayed with medium containing 1 μg/ml of cycloheximide and serial 2-fold dilutions of the test drugs. After incubation at 35° C. for 72 h, cultures were fixed and stained for inclusions with fluorescein-conjugated antibody to the chlamydial lipopolysaccharide genus-specific antigen (Pathfinder; Biorad, Redmond, Wash.). The minimum inhibitory concentration (MIC) was the lowest antibiotic concentration at which no inclusions were seen. The minimal bactericidal concentration (MBC) was determined by aspirating the antibiotic-containing medium, washing wells twice with phosphate-buffered saline, and adding antibiotic-free medium. The infected cells were frozen at −70° C., thawed, passed onto new cells, incubated for 72 h, and then fixed and stained as described above. The MBC was the lowest antibiotic concentration that resulted in no inclusions after passage. All tests were run in duplicate (Roblin, P. M. et al., “In vitro activity of CEM-101, a new fluoroketolide antibiotic, against Chlamydia trachomatis and Chlamydia (Chlamydophila) pneumonia,” Antimicrob Agents Chemother., Vol. 54, No. 3, pp. 1358-1359 (2010)). The MIC and MIB results of the study for C. trachomatis and C. pneumoniae are shown in Tables 5 and 6.

TABLE 5
Activities of the Active and Other Antibiotics against
10 Isolates of C. trachomatis

| | MIC (μg/mL) | | | MBC (μg/mL) | |
|---|---|---|---|---|---|
| Drug | Range | 50% | 90% | Range | 90% |
| The Active | 0.03-0.125 | 0.06 | 0.125 | 0.03-0.125 | 0.125 |
| Levofloxacin | 0.125-0.5 | 0.25 | 0.25 | 0.125-1 | 0.5 |
| Doxycycline | 0.03-0.25 | 0.06 | 0.125 | 0.03-0.25 | 0.125 |
| Azithromycin | 0.003-0.03 | 0.0075 | 0.015 | 0.007-0.03 | 0.015 |

TABLE 6
Activities of the Active and Other Antibiotics against 10
Isolates of C. pneumoniae

| | MIC (μg/mL) | | | MBC (μg/mL) | |
|---|---|---|---|---|---|
| Drug | Range | 50% | 90% | Range | 90% |
| The Active | 0.125-0.5 | 0.25 | 0.5 | 0.125-0.5 | 0.25 |
| Levofloxacin | 0.5 | 0.5 | 0.5 | 0.125-2 | 2 |
| Doxycycline | 0.0625-0.125 | 0.125 | 0.125 | 0.25-0.5 | 0.5 |
| Azithro- | 0.03-0.0625 | 0.0625 | 0.0625 | 0.0625-0.25 | 0.25 |
| mycin | | | | | |

For the active, the MIC at which 90% of the isolates of C. trachomatis were inhibited was 0.125 μg/ml (range 0.03-0.125 μg/ml). MIC₉₀ of the isolates of C. pneumoniae was 0.5 μg/ml (range 0.125-0.5 μg/ml). The minimal bactericidal concentrations at which 90% of the isolates were killed by the active (MBC₉₀) were 0.125 μg/ml for C. trachomatis (range 0.03-0.125 μg/ml) and 0.25 for C. pneumoniae (range 0.125-0.5 μg/ml). The MBC₉₀s for levofloxacin, doxycycline, and azithromycin were 2, 0.5, and 0.25 μg/ml, respectively.

Therefore, these results show that the in vitro activity of the active against C. trachomatis and C. pneumoniae was comparable to doxycycline.

### Mechanism of Action

For the studies below, samples were prepared with bis hydrochloride salt, and the data is expressed based on the free base (“the active”). The mechanism of action of the Crystalline Mono Hydrochloride Salt was determined by two different approaches via study of the active, as described below.

In the first approach, Antibacterial Mechanism of Action: In Vitro Inhibition of Bacterial Transcription and Translation, the ability of the active to inhibit bacterial protein synthesis was assessed using an in vitro cell-free bacterial transcription and translation assay (commercially-available from Promega Corporation, Madison, Wis.) (Beckler, G., Promega Notes 31 (1991) pp. 3-6). The active inhibited the synthesis of reporter protein with an IC₅₀ of 8.3±0.18 μM. This value was comparable to the IC₅₀ values determined for the comparator tetracyclines, doxycycline and minocycline (IC₅₀ values of 4.7±0.48 and 2.4±0.22 μM, respectively). These results provide evidence that the active functions as a classical tetracycline by inhibiting bacterial protein synthesis.

In the second approach, Antibacterial Mechanism of Action: Inhibition of Macromolecular Synthesis in Staphylococcus aureus, the ability of the active to target bacterial protein synthesis was further confirmed in a whole-cell assay of macromolecular synthesis in the Gram-positive organism, S. aureus. The active inhibited, in a dose-dependent manner, the incorporation of [³H]-leucine into proteins of the growing organism within the concentration range of 0.25-8 fold the MIC (0.063-2 μg/mL). A maximum inhibition of 80% was observed at 8-fold the MIC which was comparable to the values obtained for the tetracycline comparators doxycycline and minocycline. In contrast, the active at 8-fold the MIC demonstrated less than 20% inhibition for the synthesis of cell wall, DNA, RNA and lipid components of the test bacteria. The results of this study indicate that the active acts as a selective inhibitor of bacterial protein synthesis at concentrations comparable to known tetracyclines.

The in vitro susceptibility studies described above included tetracycline-resistant strains with characterized tetracycline resistance genes. Strains were selected that harbored the most common tetracycline resistance genes: efflux (tetK, tet38, tetL, tetS, tetB, and tetD), ribosomal protection (tetM and tetO), as well as P. acnes resistant by rRNA point mutation. The MIC values for these selected strains demonstrated a degree of cross-resistance between the active and other tetracyclines, as shown in Table 7. The presence of a tetracycline resistance gene increased the MIC of the active relative to susceptible strains (with the exception of tetK in S. aureus), with MIC values similar to those of doxycycline and/or minocycline, but generally lower than those of tetracycline.

TABLE 7
Activity of the Active Against Bacterial Strains with
Characterized Tetracycline Resistance Mechanisms.

| | | | | Doxy- | Mino- |
|---|---|---|---|---|---|
| | | | The Active | cycline | cycline |
| | Strain | Mech./ | MIC | MIC | MIC |
| Organism | PBS # | Genotype | (μg/mL) | (μg/mL) | (μg/mL) |
| P. acnes | 1073 | 16S rRNA | >8 | 8 | 2 |
| | | point | | | |
| | | mutation | | | |
| S. aureus | 1739 | tet38 | 4 | 2 | 0.5 |
| E. coli | 669 | tetB | >64 | 64 | 16 |
| K. pneumoniae | 266 | tetD | >64 | 64 | 64 |
| S. aureus | 1309 | tetK | 0.5 | 2 | 0.5 |
| E. faecium | 1323 | tetK | 8 | 4 | 2 |
| E. faecalis | 274 | tetL | 32 | 16 | 16 |
| S. aureus | 1310 | tetM | 8 | 16 | 4 |
| S. pyogenes | 792 | tetM | 4 | 4 | 4 |
| S. agalactiae | 897 | tetM | 16 | 8 | 16 |
| S. pneumoniae | 511 | tetM | 4 | 4 | 8 |
| E. faecalis | 276 | tetM | 16 | 8 | 16 |
| E. faecium | 965 | tetM | 8 | 4 | 8 |
| S. pyogenes | 330 | tetO | 16 | 8 | 8 |
| S. agalactiae | 316 | tetO | 32 | 8 | 16 |
| E. faecium | 1324 | tetO | 16 | 4 | 2 |
| E. faecalis | 949 | tetS | 8 | 2 | 4 |

Numerous alterations, modifications, and variations of the preferred embodiments disclosed herein will be apparent to those skilled in the art, and they are all anticipated and contemplated to be within the spirit and scope of the claimed invention. For example, although specific embodiments have been described in detail, those with skill in the art will understand that the preceding embodiments and variations can be modified to incorporate various types of substitute, additional or alternative materials. Accordingly, even though only few variations of the present invention are described herein, it is to be understood that the practice of such additional modifications and variations and the equivalents thereof, are within the spirit and scope of the invention as defined in the following claims. All patent applications, patents, and other publications cited herein are incorporated by reference in their entirety.

What is claimed is:

1. A method for treating a methicillin resistant Staphylococcus aureus (MRSA) infection comprising administering to a subject a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt thereof.

2. The method of claim 1, wherein the pharmaceutically acceptable salt is selected from the group consisting of crystalline mono hydrochloride, crystalline mono mesylate, and crystalline mono sulfate.

3. The method of claim 2, wherein the salt is mono hydrochloride.

4. The method of claim 2, wherein the salt is mono mesylate.

5. The method of claim 2, wherein the salt is mono sulfate.

6. The method of claim 1, wherein MRSA is selected from community acquired MRSA (MRSA-CA) and hospital-acquired MRSA (MRSA-HA).

7. A method for treating a peptic ulcer comprising administering to a subject a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt thereof.

8. The method of claim 7, wherein the pharmaceutically acceptable salt is selected from the group consisting of crystalline mono hydrochloride, crystalline mono mesylate, and crystalline mono sulfate salts.

9. The method of claim 8, wherein the salt is mono hydrochloride.

10. The method of claim 8, wherein the salt is mono mesylate.

11. The method of claim 8, wherein the salt is mono sulfate.

12. The method of claim 7, wherein the method further comprises administering to the subject at least one additional active ingredient.

13. The method of claim 12, wherein the at least one additional active ingredient is selected from a proton pump inhibitor and bismuth.

14. A method for treating a Helicobacter pylori infection comprising administering to a subject a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt thereof.

15. The method of claim 14, wherein the pharmaceutically acceptable salt is selected from the group consisting of crystalline mono hydrochloride, crystalline mono mesylate, and crystalline mono sulfate.

16. The method of claim 15, wherein the salt is mono hydrochloride.

17. The method of claim 15, wherein the salt is mono mesylate.

18. The method of claim 15, wherein the salt is mono sulfate.

19. A method of treating a Chlamydia trachomatis infection comprising administering to a subject a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt thereof.

20. The method of claim 19, wherein the pharmaceutically acceptable salt is selected from the group consisting of crystalline mono hydrochloride, crystalline mono mesylate, and crystalline mono sulfate.

21. The method of claim 20, wherein the salt is mono hydrochloride.

22. The method of claim 20, wherein the salt is mono mesylate.

23. The method of claim 20, wherein the salt is mono sulfate.
